(19) United States
(12) Patent Application Publication
(10) Pub. No.: US 2016/0354417 A1
(43) Pub. Date: Dec. 8, 2016

(54) SYSTEMS, METHODS, AND COMPOSITIONS RELATING TO COMBIOMICS

(71) Applicant: MICRO-NATURE LLC, San Francisco, CA (US)

(72) Inventors: Richard Baird SMITTLE, Morgantown, WV (US); Gregory Dean SUNVOLD, Lewisburg (US); John Boyd PHELPS, Andover, NJ (US); John HOMMEYER, San Francisco, CA (US)

(73) Assignee: MICRO-NATURE LLC, San Francisco, CA (US)

(21) Appl. No.: 15/116,510

(22) PCT Filed: Feb. 4, 2015
(86) PCT No.: PCT/US2015/014522

§ 371 Date: Aug. 3, 2016

Related U.S. Application Data
(60) Provisional application No. 61/935,402, filed on Feb. 4, 2014.

(51) Int. Cl. A61K 35/744; A23K 20/10; A23K 10/18; A61K 35/747; A23L 33/21; A61K 9/00; A61K 31/7004; A61K 36/28; A23L 3/3571; A23L 33/135

(52) U.S. Cl. CPC A61K 35/744; A23L 3/3571; A23K 20/10; A23K 10/18; A23L 33/135; A23L 33/21; A61K 9/0014; A61K 31/7004; A61K 36/28; A61K 35/747; A23V 2002/00; A23Y 2280/15; A23Y 2280/55; A23Y 2220/67; A61K 2035/115

(57) ABSTRACT

A process for producing a combiomic complex is disclosed. The process includes: (i) obtaining a pre-combiomic mixture, which includes at least one non-adhered bacteria and/or yeast and at least one non-adhered prebiotic; and (ii) treating at least some of the pre-combiomic mixture to form a combiomic mixture, which comprises a combiomic complex that includes at least one bacteria and/or yeast adhered to at least one prebiotic.

### RELATED CASE

This application claims priority to U.S. provisional application No. 61/935,402, filed Feb. 4, 2014, and is incorporated herein by reference for all purposes.

### FIELD

The present teachings relate generally to systems, methods, and compositions that promote human and animal health, and facilitate food safety and food preservation. More particularly, the present teachings relate to systems, methods, and compositions for making and using a combiomic complex to promote human and animal health and to facilitate food safety and food preservation.

### BACKGROUND

Microorganisms, including bacteria and yeasts, are increasingly being explored and exploited as solutions to many current health and sanitation problems. However, due to their unique nature, many of their potential applications to human and animal health are still not well understood. Microorganisms that provide health benefits to the host are generally referred to as probiotics.

Unfortunately, conventional systems, methods, and compositions used to deliver bacteria and/or yeast suffer from certain drawbacks. In particular, probiotics have difficulty surviving the harsh environments of a host's digestive system. By way of example, the high concentration of acids in the stomach that produce a low pH therein tends to deactivate bacterial cells. Similarly, bile acids secreted into the duodenum to help emulsify dietary fat for absorption also disrupt the lipophilic components of bacterial cell membranes, further resulting in de-activation of the probiotics. Due to these complexities, the challenge remains how to deliver microorganisms and microorganism/prebiotic combinations to a host in a manner that promotes viability of those microorganisms.

### SUMMARY OF THE INVENTION

In one aspect, the present teachings disclose a process for producing a combiomic complex. The process includes: (i) obtaining a pre-combiomic mixture, which includes at least one non-adhered bacteria and/or yeast and at least one non-adhered prebiotic; (ii) treating at least some of the pre-combiomic mixture to form a combiomic mixture, which comprises a combiomic complex that includes at least one of bacteria and/or yeast adhered to at least one prebiotic. Obtaining the pre-combiomic mixture may include obtaining between about 1×10⁵ and about 1×10¹⁰ colony forming units (“CFU”) of bacteria and/or yeast per gram of the non-adhered prebiotic. Preferably, treating facilitates secretion of a biofilm by the non-adhered bacteria and/or yeast, such that the biofilm promotes adhering of the non-adhered bacteria and/or yeast to the non-adhered prebiotic.

According to one embodiment of the present teachings, the process includes removing the combiomic complex from the combiomic mixture. The amount of combiomic complex may be between about 0.0001% and about 10% by weight of combiomic mixture. In one embodiment of the present teachings, the process includes placing substantially non-fermenting bacteria and/or yeast in the combiomic complex. In another embodiment of the present teachings, the process includes drying the combiomic complex.

In another aspect, the present teachings disclose a combiomic complex composition, which includes a combiomic complex that has at least one bacteria and/or yeast adhering to at least one prebiotic, and the bacteria and/or yeast in the combiomic complex composition is in a substantially non-fermenting state. The combiomic complex may be free of digestive enzymes found in a human or animal. The combiomic complex may also not be in contact with food.

According to one embodiment of the present teachings, the combiomic complex further includes a biofilm that is produced during growth of at least one of the bacteria and/or yeast and facilitates adhesion the bacteria and/or yeast to at least one of the prebiotic. In one embodiment of the present teachings, the biofilm is an exopolysaccharide. In another embodiment of the present teachings, the biofilm includes at least one member chosen from a group comprising galacturonic acid, glucuronic acid, and mannuronic acid. In yet another embodiment of the present teachings, the biofilm comprises a glycocalyx matrix that surrounds at least one of the bacteria and/or yeast to facilitate adhesion of at least one bacteria and/or yeast to at least one prebiotic.

The combiomic complex may include a bacteria and/or yeast that promotes food safety and/or food preservation that is at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Saccharomyces cerevisiae, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophiles, Bacillus cereus, Propionibacterium freundenreichii, and Oxalobacter formagenes. The combiomic complex also includes a prebiotic that may be at least one member chosen from a group comprising fructooligosaccharides (FOS), short-chain FOS, galactooligosaccharides, xylooligosaccharides, oligo derivatives from starch, inulin, chicory, soy oligosaccharides, trehalose, raffinose, stachyose, lactosucrose, lactulose, apple pomace, paw paw, galacto-oligosaccharides, soybean oligosaccharides, gluco-oligosaccharides, cyclodextrins, gentio-oligosaccharides, germinated barley foodstuffs, oligodextrans, pecti-oligosaccharides, mannan-oligosaccharides, lactose, resistant starches, oligo-saccharides, oligo-saccharides from melobiose, n-acetylchito-oligosaccharides, shrimp shells, polydextrose, sugar alcohols, konjac glucomannan, whole grain, corn steep, Jerusalem Artichoke, wheat bran, rice bran, plantain, bananas, apple pulp, B-glucan, carboxymethylcellulose (CMC), methylcellulose, hydroxypropylmethylcellulose (HPMC), psyllium, guar gum, citrus pectin, pectin, xanthan gum, gum Arabic, gum talha, and alginate. A combiomic size ratio, i.e., a ratio of a particle size of the adhering prebiotic to the individual cell size of the bacteria and/or yeast, may be between about 200:1 and about 8000:1. The combiomic complex composition may also include a fluid, such that the concentration of the combiomic complex in the combiomic complex composition is a value that is between about 0.0001% and about 5% by weight of the combiomic complex composition.

According to one embodiment of the present teachings, the combiomic complex composition may be in a state that is at least one member chosen from a group comprising solid, dried, freeze-dried, powder, granule, fluid, gel, liquid, and syrup. Further, the combiomic complex composition may include at least one member chosen from a group comprising palatant, flavor enhancer, food, fat-enriched food or medicine, binder, preservative, energy source, beverage, supplement, pill, a coating, and food coating. The energy source may be at least one member chosen from a group comprising apple juice, apple juice concentrate, dextrose, dextrose monohydrate, dextrose hydride, grape sugar, D-glucose, corn sugar, corn syrup solids, high fructose corn syrup, levulose, invert sugar, glucose, galactose, xylose, ribose, mannose, sorbose, high fructose corn syrup, apple pulp, honey, sugar, maple syrup, pear juice, grape juice, orange juice, and fruit juice. The preservative includes at least one member chosen from a group comprising glycerol, dextrose, vitamin E, milk solids, sugar concentrates, propylene glycol, dimethyl sulfoxide (DMSO), mannitol, sorbitol, casein, meat concentrates, humectants, non-ionizing compounds that include many humectants, glycine betaine, sugars, sucrose, fructose, galactose, lactose, ethylene glycol, erythritol, threitol, dimethylformamide, 2-methyl-2,4-pentanediol, trehalose, tween 80, and capsular material. An amount of preservative present in combiomic complex may range from between about 30% by weight of the combiomic complex composition to about 70% by weight of the combiomic complex composition.

According to one embodiment of the present teachings, the combiomic complex composition may further include a carrier for conveying the combiomic complex composition to a human or an animal. A carrier may be at least one member chosen from a group comprising capsule, sachet, pill, human food, pet food, pet treat, supplement, meat, dairy product, vegetable, fruit, fermented beverage, topical application, wound dressing, sunscreen, makeup product, mascara, cream, mouthwash, tampon, feminine pad, and dentifrice. A carrier may also include at least about 1×10⁵ cfu per gram of bacteria and/or yeast in the combiomic complex composition.

The combiomic complex composition may include one species of bacteria or yeast that promotes human or animal health and/or another species that promotes pathogen control in human or animal food. The bacteria or yeast that promotes human or animal health may include at least one member chosen from a group Lactobacillus, Bifidobacteria, Lactobacillus plantarum, Lactobacillus acidiophilis, Lactobacillus reuterii, Bifidobacterium bifidus, and Bifidobacterium animalis. The bacteria and/or yeast that promotes pathogen control in a human or animal food may include at least one member chosen from a group comprising Pediococci, Pediococcus pentosaceus, Pediococcus acidilactici, Lactococcus lactis, Lactococcus cremoris, Lactobacillus plantarum, Lactobacillus acidiophilis, Lactobacillus reuterii, L. bulgaricus, L. cuvatus, L. sakaii, L. fermentum, and Enterococcus faecium. Preferably, the combiomic complex composition is shelf-stable for a time that is at least about 6 months.

In yet another aspect, the present teachings disclose a pre-combiomic composition. The composition includes: (i) at least one bacteria and/or yeast; (ii) at least one prebiotic that is not adhering to the bacteria and/or yeast; and wherein the concentration of at least one prebiotic is between about 0.0001% and about 2%, by weight, of pre-combiomic composition, and wherein the concentration of at least one of the bacteria and/or yeast has between about 1×10⁵ and about 1×10⁸ cfu/g of the pre-combiomic composition. The pre-combiomic composition may further include a growth medium and a culture energy source.

In yet another aspect, the present teachings disclose a method for promoting human or animal health, which includes (i) providing a combiomic complex that includes at least one bacteria and/or yeast adhering to at least one prebiotic, wherein at least one of the bacteria and/or yeast in the combiomic complex composition is in a substantially non-fermenting state; and (ii) directing use of the combiomic complex by a human or an animal. Promoting human or animal health may include treating at least one health condition chosen from a group comprising heart disease, dental caries, intestinal disorder, irritable bowel syndrome, diarrhea, acne, skin wrinkles, skin disease, gum disease, plaque formation, gum infection, inflammation, and improving healing during tooth removal. According to another embodiment of the present teachings, directing facilitates development of microbiota in at least one member chosen from a group comprising mouth, intestine, and colon, to promote human and/or animal health. According to one embodiment of the present teachings, the bacteria and/or yeast that adheres to a prebiotic is more resistant than non-adhered bacteria and/or yeast to acid exposure.

According to another embodiment of the present teachings, providing includes preparing a topical application that includes the combiomic complex, and directing includes providing instructions for applying the topical application on a surface of the human or animal. The topical may be at least one member chosen from a group comprising spray, wound dressing, sunscreen, makeup product, mascara, cream, mouthwash, tampon, feminine pad, and dentifrice. The surface of the human or animal may include at least one surface of a member chosen from a group comprising tooth, gum, skin, mucous membrane, and ear canal.

In yet another aspect, the present teachings disclose a method for promoting pathogen control in food. The method includes: (i) obtaining a food item; applying to the food item a combiomic complex to form a combiomic food product; (ii) incubating the combiomic food product to form a substantially pathogen free food; and wherein the combiomic complex includes at least one bacteria and/or yeast that adheres to at least one prebiotic by virtue of a biofilm produced by at least one of the bacteria and/or yeast. Food may be at least one member chosen from a group comprising ice cream, yogurt, milk, meat, fermented meat, kibbled food, kibble, an expanded food, pelleted food, extruded food, refrigerated food, refrigerated treat, frozen food, frozen treat, biscuit, raw food, fried food or treat, soft-moist food, pellet, fine, broken piece of food, jerky-style treat, injection-molded treat, treat, supplement, salad ingredient, ground fruit or vegetable, meal, slaughtered carcass, piece or chunk of meat, fabricated meat, fabricated protein chunk, livestock feed, steam-flaked feed, and aquaculture feed.

In yet another aspect, the present teachings disclose a method for producing a shelf-stable food. The method includes: (i) obtaining a food item; (ii) applying to the food item a combiomic complex to form a combiomic food product; (iii) incubating the combiomic food product to form a substantially shelf-stable food; and wherein the combiomic complex includes at least one bacteria and/or yeast that adheres to at least one prebiotic by virtue of a biofilm produced by at least one of the bacteria and/or yeast.

### BRIEF DESCRIPTION OF THE DRAWINGS

FIG. 1 is an electron micrograph of a combiomic complex, according to one embodiment of the present teachings and that includes a biofilm for adhering Pediococcus acidilactici and Pediococcus pentosaceus to ground chicory.

FIG. 2 is a flowchart showing certain salient steps used in a process for producing a combiomic complex, according to one embodiment of the present teachings.

FIG. 3 is a flowchart showing certain salient steps that involve a combiomic complex used in a process for promoting human or animal health, according to one embodiment of the present teachings.

FIG. 4 is a flowchart showing certain salient steps that involve a combiomic complex used in a process for promoting food safety and/or food preservation, according to one embodiment of the present teachings.

FIG. 5 is a bar graph showing an amount of bacteria in three different settings, i.e., in a combiomic complex, in a synbiotic mixture and as a probiotic alone, that is initially untreated and then after treatment with hydrochloric acid (“HCl”) for a duration of about 30 minutes.

FIG. 6 is a graph of pH of chicken meat and an amount of E. coli versus time.

### DETAILED DESCRIPTION OF THE PREFERRED EMBODIMENTS

In the following description numerous specific details are set forth in order to provide a thorough understanding of the present teachings. It will be apparent, however, to one skilled in the art that the present teachings may be practiced without limitation to some or all of these specific details. In other instances, well known process steps have not been described in detail in order to not unnecessarily obscure the present teachings.

The present teachings describe use of a “combiomic complex” to deliver microorganisms to a host or to a food product. A “combiomic complex” refers to a complex that includes a bacteria and/or yeast adhering to a prebiotic. In sharp contrast, prebiotics and bacteria are conventionally provided (e.g., in items such as supplements, dietary rations, feeds, foods, and beverages) as separate ingredients, or as mixed ingredients that do not adhere to each other because they are typically not provided in conditions that may facilitate their adherence to each other (e.g., moisture greater than 15% and temperature greater than 90° F.).

In the combiomic complex of the present teachings, the bacteria and/or yeast may be selected to serve many different beneficial functions. As will be explained below, the bacteria and/or yeast component so selected, may allow the resulting combiomic complex of the present teachings to serve one or more functions chosen from a group comprising promoting health, controlling pathogens, and preserving food. When certain bacteria and/or yeast, in a combiomic complex composition, are administered to promote a human's or an animal's health, then that bacteria is referred to herein as a “health-promoting” bacteria and/or yeast. In one aspect of the present teachings, health-promoting bacteria and/or yeast include at least one member chosen from a group comprising Lactobacillus, Bifidobacteria, Lactobacillus plantarum, Lactobacillus acidiophilis, Lactobacillus reuterii, Bifidobacterium bifidus, Bifidobacterium animalis, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Pediococcus acidilactici, Pediococcus pentosaceus. Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium thermophilium, Enterococcus faecalis, Streptococcus cremoris, Streptococcus salivarius, Streptococcus diacetylactis, Streptococcus intermedius, Lactobacillus paracasei, Pediococcus acidilactici, Pediococcus pentosaceus, Streptococcus thermophiles, Streptococcus salivarius subsp. Thermophilus, Bacillus cereus, Proprionibacteria freundenreichii, Bacillus coagulans (L. sporegenes), and Oxalobacter formagenes.

In a combiomic complex, a bacteria and/or yeast that promotes pathogen control is referred herein to as a “pathogen control” bacteria and/or yeast. A pathogen control bacteria and/or yeast may be at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, and Oxalobacter formagenes.

As used herein, “pathogen control” means substantially killing or inhibiting the growth of at least one pathogen. Examples of a pathogen include Salmonella, pathogenic Escherichia coli, Shigella, Listeria monocytogenes, Staphylococcus aureus, Campylobacter spp, pathogenic Esherichia coli, Staphylococcus auerus, Campylobacter jejuni, Clostridium difficile, Campylobacter coli, Clostridium botulinum, Clostridium perfringens, Coxeilla burnetii, Trichinella spiralis, Vibrio parahaemolyticus, and Vibrio cholera. According to one embodiment of the present teachings, substantially killing or inhibiting the growth of at least one pathogen means reducing pathogen growth and/or survival on a food product by at least about 1,000 colony forming units of a pathogen per gram of the food product (expressed as unit of “cfu/g”). In one aspect of the present teachings, such killing or growth inhibition may be carried out by treating a food inoculated with a combiomic complex for about 48 hours at about 22° C.

The present teachings also recognize that a bacteria and/or yeast may promote food preservation when a combiomic complex composition containing the bacteria and/or yeast is applied to a food product. Accordingly, such bacteria and/or yeast is referred herein to as a “food preservation” bacteria. According to certain embodiments of the present teachings, a food preservation bacteria used in a combiomic complex is the same as a pathogen control bacteria. In other words, a bacteria used in a combiomic complex to promote pathogen control may also, or alternatively, be used to promote food preservation, i.e., by substantially killing and/or inhibiting the growth of at least one food-spoilage microorganism. Substantially killing and/or inhibiting the growth and/or survival of at least one food-spoilage microorganism means reducing the growth and/or survival of one more food-spoilage microorganisms on the food product by at least about 1,000 cfu/g.

Food spoilage organisms may be described physiologically using such intrinsic and descriptive terms as aciduric, acidophilic, refrigerated, mesophilic, xerotolerant, intermediate moisture, anaerobic spore formers, temperature psychrophiles, temperature thermophilic, halophilic, and osmotolerant. A food spoilage microorganism may be at least one member chosen from a group comprising Rhizopus nigricans, Penicillum, Aspergillus niger, Bacillus subtilis, Enterobacter aerogenes, Saccharomyces, Zygosaccharomyces, Micrococcus roseus, Aspergillus, Rhizopus, Erwinia, Botrytis, Rhodotorula, Alcaligenes, Clostridium, Proteus vulgaris, Pseudomonas fluorescens, Micrococcus, Lactobacillus, Leuconostoc, Alcaligenes, Flavobacterium, Proteus, and Acetobacter.

In certain embodiments of the present teachings, a combiomic complex composition includes at least one type of bacteria and/or yeast chosen from a group comprising human or animal health-promoting bacteria, pathogen control bacteria, and food preservation bacteria. Each of different types of combinations that include human or animal health-promoting bacteria, pathogen control bacteria, and food preservation bacteria, allows the resulting combiomic complex to provide many benefits. By way of example, a combiomic complex may include one or more bacteria and/or yeast that facilitate pathogen control and food preservation, to produce a food that is safe and shelf-stable. That food may also include, in the combiomic complex, health-promoting bacteria, such that when the food is consumed, the health benefits of the health-promoting bacteria are delivered to a host, in a food that is both safe and shelf-stable.

The present teachings recognize that size of a bacteria and/or yeast cell in the combiomic complex may impact the bacteria or yeast's efficacy in promoting health, controlling pathogens, and preserving food. To this end, it is noteworthy that the individual cell size of a bacteria and/or yeast (which is the diameter of a bacteria and/or yeast cell) in a combiomic complex composition may vary widely. According to one embodiment of the present teachings, the individual bacteria and/or yeast cell size, in a combiomic complex composition, is a value that is between about 0.25 microns and about 1.0 micron. According to another embodiment of the present teachings, the individual bacteria and/or yeast cell size, in a combiomic complex composition, is a value between about 1.0 microns and about 1.5 microns. According to yet another embodiment of the present teachings, the individual bacteria and/or yeast cell size, in a combiomic complex composition, is a value between about 1.5 microns and about 2.0 microns. According to yet another embodiment of the present teachings, the individual bacteria and/or yeast cell size, in a combiomic complex composition, is a value between about 2.0 microns and about 4.0 microns. According to a preferred embodiment of the present teachings, the individual bacteria and/or yeast cell size, in a combiomic complex composition, is a value between about 0.5 microns and about 1.0 microns. According to another preferred embodiment of the present teachings, the individual bacteria and/or yeast cell size, in a combiomic complex composition, is a value between about 1.0 microns and about 2.0 microns.

The bacteria and/or yeast, in a host environment or when applied to food products, deactivates over time. In the combiomic complex composition of the present teachings, however, a prebiotic component adhering to the beneficial bacteria and/or yeast serves as a valuable fuel source for the bacteria and/or yeast in its metabolically active state. In a hostile environment, which may be encountered inside a digestive tract of a human or animal, a prebiotic component facilitates metabolic activity of beneficial bacteria and/or yeast. As a result, the adhering prebiotic component allows the combiomic complex of the present teachings to convey a significant dose of a potentially metabolically active bacteria and/or yeast inside a hostile environment.

In the combiomic complex, the prebiotic component is a composition that selectively stimulates growth or activity of one or more bacteria (e.g., beneficial bacteria). In one aspect, a prebiotic may act as non-digestible fiber source that selectively stimulates the growth or activity of certain probiotics (e.g., bifidobacteria or lactic acid bacteria), in preference to pathogenic bacteria (e.g., clostridia or salmonella). One example of a non-digestible fiber source is a viscous fiber, which may be the form of a gel when appropriately hydrated. The term “viscous fiber,” as used herein, refers to one type of a prebiotic. According to one embodiment of the present teachings, a prebiotic includes at least one member chosen from a group comprising fructooligosaccharides (FOS), short-chain FOS, galactooligosaccharides, xylooligosaccharides, oligo derivatives from starch, inulin, chicory, soy oligosaccharides, trehalose, raffinose, stachyose, lactosucrose, lactulose, apple pomace, paw paw, galacto-oligosaccharides, soybean oligosaccharides, gluco-oligosaccharides, cyclodextrins, gentio-oligosaccharides, germinated barley foodstuffs, oligodextrans, pecti-oligosaccharides, mannan-oligosaccharides, lactose, resistant starches, oligo-saccharides, oligo-saccharides from melobiose, n-acetylchito-oligosaccharides, shrimp shells, polydextrose, sugar alcohols, konjac glucomannan, whole grain, corn steep, Jerusalem Artichoke, wheat bran, rice bran, plantain, bananas, apple pulp, B-glucan, carboxymethylcellulose (CMC), methylcellulose, hydroxypropylmethylcellulose (HPMC), psyllium, guar gum, citrus pectin, pectin, xanthan gum, gum Arabic, gum talha, and alginate.

In a combiomic complex of the present teachings, the prebiotic component is preferably ground, pulverized, or granulated to reduce the prebiotic's particle size. To the extent such methods result in particles that are larger than desired, such larger particles may be screened or filtered out prior to use. A reduced particle size provides the advantage of a greater prebiotic surface area, to which a larger number of bacteria and/or yeast may adhere.

Against this backdrop of reduced prebiotic particle size, the size of the adhering bacteria and/or yeast is preferably relatively smaller. The present teachings recognize that the magnitude of the prebiotic size relative to the bacteria and/or yeast provides protection to the bacteria and/or yeast when it passes through various parts of the intestinal tract. By way of example, the individual cell size of the bacteria and/or yeast may be expressed in terms of “per particle size of the adhering prebiotic source.” According to the present teachings, this parameter may be referred to as the “Combiomic Size Ratio.” The Combiomic Size Ratio is the ratio of a particle size of the adhering prebiotic source to the individual cell size of the bacteria and/or yeast source.

In one embodiment of the present teachings, a Combiomic Size Ratio of a combiomic complex has a value that is between about 200 and about 400. In another embodiment of the present teachings, a Combiomic Size Ratio of a combiomic complex has a value that is between about 200 and about 400. In yet another embodiment of the present teachings, a Combiomic Size Ratio of a combiomic complex has a value that is between about 400 and about 800. In yet another embodiment of the present teachings, a Combiomic Size Ratio of a combiomic complex has a value that is between about 800 and about 1,200. In yet another embodiment of the present teachings, a Combiomic Size Ratio of a combiomic complex has a value that is between about 1,200 and about 2,400. In yet another embodiment of the present teachings, a Combiomic Size Ratio of a combiomic complex has a value that is between about 2,400 and about 4,000. In yet another embodiment of the present teachings, a Combiomic Size Ratio of a combiomic complex has a value that is between about 4,000 and about 6,000. In yet another embodiment of the present teachings, a Combiomic Size Ratio of a combiomic complex has a value that is between about 6,000 and about 8,000. In yet other embodiments of the present teachings, a Combiomic Size Ratio of a combiomic complex has a value that is between about 800 and about 1,200. In yet another embodiments of the present teachings, a Combiomic Size Ratio of a combiomic complex has a value that is between about 1,200 and about 2,400.

The present teachings recognize that the bulk density of a prebiotic, i.e., the mass of prebiotic per unit volume of pre-combiomic mixture, may be significant in facilitating adherence of a bacteria and/or yeast to a prebiotic (i.e., in a combiomic mixture). The term “pre-combiomic,” as used herein and according to the present teachings, refers to a non-adhered state of bacteria and/or yeast and a prebiotic component. According to the present teachings, the greater the bulk density of the prebiotic in the pre-combiomic mixture, the more likely it may be that the prebiotic will act as a substrate for adherence of a bacteria and/or yeast because the greater bulk density results in more contact between prebiotic and bacteria and/or yeast particles. According to one embodiment of the present teachings, the bulk density of a prebiotic in a pre-combiomic mixture is between 350 g/L and about 400 g/L. According to another embodiment of the present teachings, the bulk density of a prebiotic in a pre-combiomic mixture is between about 400 g/L and about 450 g/L. According to yet another embodiment of the present teaching, the bulk density of a prebiotic in a pre-combiomic mixture is between about 450 g/L and about 500 g/L. According to another embodiment of the present teaching, the bulk density of a prebiotic in a pre-combiomic mixture is between about 350 g/L and about 425 g/L. According to another embodiment of the present teaching, the bulk density of a prebiotic in a pre-combiomic mixture is between about 375 g/L and about 500 g/L. According to another embodiment of the present teaching, the bulk density of a prebiotic in a pre-combiomic mixture is between about 350 g/L and about 450 g/L.

In one embodiment, the adhesion of bacteria and/or yeast with the prebiotic component is accomplished by growing the bacteria and/or yeast on the prebiotic. In preferred embodiments of the present teachings, such adhesion is effected prior to the addition to the food/feed/beverage product. In sharp contrast, such adhesion does not comport with conventional wisdom, as there is an expectation that bacteria and/or yeast would use the prebiotic as an energy source and then die. Further, the improved survival benefit of bacteria and/or yeast adhering to a prebiotic inside the digestive tract is currently not known, and therefore, not exploited.

In one preferred embodiment of the present teachings, the combiomic complex includes a “biofilm,” which effectively adheres the bacteria and/or yeast to the prebiotic component. The biofilm may include at least one member chosen from a group comprising acid mucin, galacturonic acid, glucuronic acid, mannuronic acid, polysaccharide, glycoprotein, and lipopolysaccharide. The biofilm is, preferably, an exopolysaccharide.

The present teachings contemplate various arrangements of the biofilm in the combiomic complex. According to one preferred arrangement, the biofilm includes a glycocalyx matrix that surrounds the adhering structure of bacteria and/or yeast and the prebiotic. In another preferred arrangement, the biofilm substantially surrounds the entire combiomic complex.

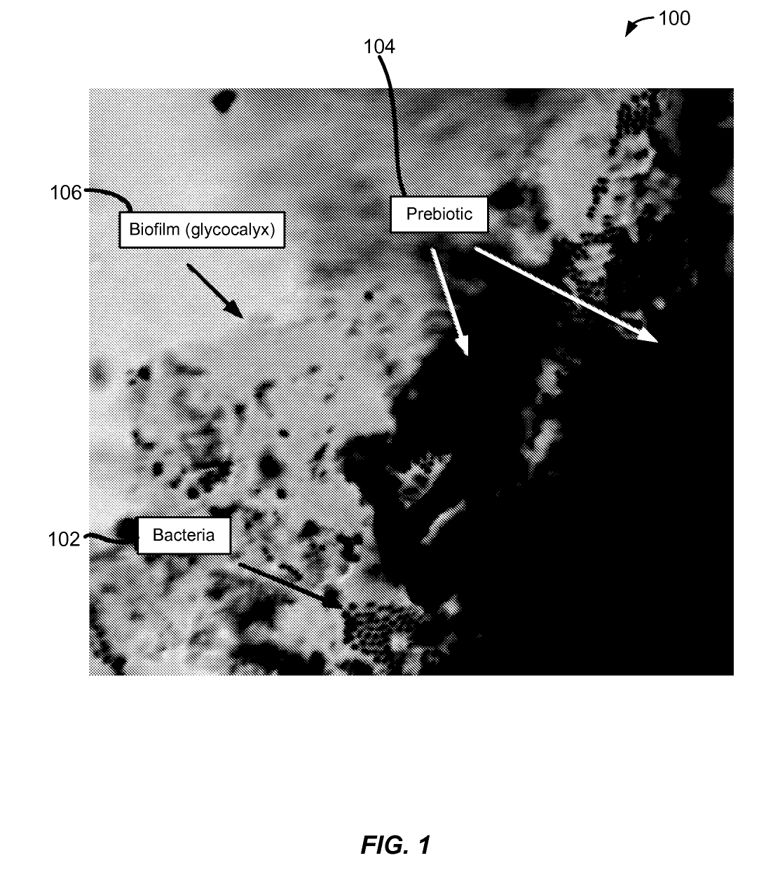

FIG. 1 is a photomicrograph 100 (at 1000× magnification) of a combiomic complex, according to one preferred embodiment of the present teachings, surrounded by a biofilm. Photomicrograph 100 shows a bacteria 102 adhered to a prebiotic 104 to form a bacteria/prebiotic complex. Further, a biofilm 106 surrounds the bacteria/prebiotic complex. Although yeast is not shown in FIG. 1, the present teachings recognize that yeast may be used instead of or in addition to bacteria 102.

To produce the combiomic complex shown in FIG. 1, bacteria (i.e., Pediococcus acidilactici and Pediococcus pentosaceus) were incubated for eight hours at about 41° C. and about 43° C. in a growth medium of beef broth, an energy source of apple juice concentrate, and ground chicory as the prebiotic. In photomicrograph 100 of FIG. 1, Pediococci bacteria is disposed within a biofilm matrix (glycocalyx) and adheres to the chicory in circular clusters. The biofilm matrix consists of glycoproteins and polysaccharides.

In one embodiment of the present teaching, the biofilm is produced during incubation or fermentation of the bacteria and/or yeast. A combiomic mixture, composed of a plurality of combiomic mixes, may include a combiomic complex and residual material (i.e., some pre-combiomic mixture) that did not transform to a combiomic complex during incubation or fermentation of the pre-combiomic mixture. Examples of such residual material include non-adhered bacteria and/or yeast, non-adhered prebiotic, fluid, an energy source, or other components associated with a combiomic complex.

As explained in further detail below, using a combiomic complex to promote health and/or food safety and preservation provides advantages over conventional techniques, including those that rely on synbiotics (which refers to a mixture of non-adhered probiotic and prebiotic). In particular, a biofilm that adheres a microorganism to a prebiotic in a combiomic complex protects both the microorganism and the prebiotic from death or degradation in a human or animal digestive tract, or during long-term storage of the combiomic complex. This results in a more effective delivery of beneficial bacteria to a host organism. Further, the prebiotic, that is also protected in a combiomic complex, provides an energy source for the activity of beneficial bacteria when metabolizing in a human or animal (e.g., after delivery of a combiomic through the host's digestive tract).

The present teachings recognize that a “biofilm” is secreted during growth or metabolism of a bacteria and/or yeast, including during fermentation of that bacteria, allowing the bacteria to attach to a surface. As a biofilm is secreted, a bacteria and/or yeast identifies a prebiotic via chemotaxis. The biofilm may generally be a mucopolysaccharide and as such, the biofilm may be thought of as relatively “sticky.” When a bacteria that includes a secreted biofilm contacts a prebiotic surface, the bacteria will adhere to the prebiotic surface.

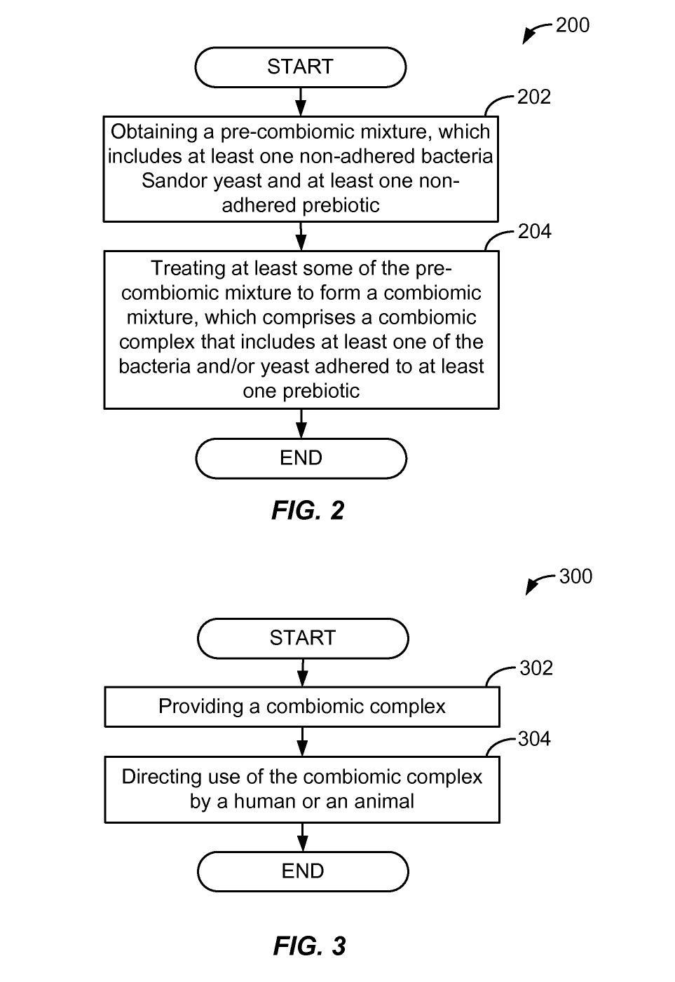

A combiomic complex composition may also include a fluid portion. The fluid may be a residual fluid (e.g., a growth media) used during a process of producing a combiomic complex (e.g., as explained below with reference to FIG. 2). Alternatively, a fluid may be added to a combiomic complex (e.g., water or a buffered solution). According to one embodiment of the present teachings, a combiomic complex is between about 0.0001%, by weight, of a combiomic complex composition and about 5%, by weight of a combiomic complex composition. In other words, according to this embodiment, between about 0.0001% and about 5% of the weight of a combiomic complex composition represents bacteria and/or yeast that is adhered to a prebiotic.

According to one embodiment of the present teachings, a combiomic complex composition is at least one member chosen from a group comprising solid, dried, freeze-dried, powder, fluid, gel, liquid, and syrup.

Likewise, a combiomic complex composition may include one or more additional components that facilitate its beneficial uses. According to one embodiment of the present teachings, a combiomic complex composition further includes at least one member chosen from a group comprising palatant, flavor enhancer, food, fat-enriched food or medicine, binder, preservative, energy source, beverage, supplement, pill, coating, and food coating. As explained in further detail below, such components may facilitate use of a combiomic complex to promote human or animal health, pathogen control and food preservation.

According to one embodiment of the present teachings, a preservative is added to a combiomic complex composition to facilitate long-term storage and stability thereof. A preservative may include at least one member chosen from a group comprising glycerol, dextrose, vitamin E, milk solids, sugar concentrates, propylene glycol, dimethyl sulfoxide (DMSO), mannitol, sorbitol, casein, meat concentrates, humectants, non-ionizing compounds that include many humectants, glycine betaine, sugars, sucrose, fructose, galactose, lactose, ethylene glycol, erythritol, threitol, dimethylformamide, 2-methyl-2,4-pentanediol, trehalose, tween 80, and capsular material. Such preservatives and/or stabilizing agents in a preserved combiomic complex help slow the deactivation of bacteria in the combiomic solid residue. According to one embodiment of the present teachings, a preserved combiomic complex includes about 60% by weight of a combiomic solid residue and about 40% by weight of a preservative, more preferably includes about 30% by weight of a combiomic solid residue and about 70% by weight of a combiomic solid residue, and even more preferably between about 30% by weight of a preservative and about 70% by weight of a combiomic solid residue.

According to another embodiment of the present teachings, a fat-enriched food or medicine may be added to the combiomic complex composition to facilitate the survival of bacteria and/or yeast in a human or animal intestinal tract after the combiomic complex composition is ingested.

According to yet another embodiment of the present teachings, an energy source is added to a combiomic complex composition. The energy source, when added to a combiomic complex composition, may serve to facilitate growth, fermentation, and/or metabolic activity of bacteria that is delivered to a human or animal host. An energy source may be at least one member chosen from a group comprising apple juice, apple juice concentrate, dextrose, dextrose monohydrate, dextrose hydride, grape sugar, D-glucose, corn sugar, corn syrup solids, high fructose corn syrup, levulose, invert sugar, glucose, galactose, xylose, ribose, mannose, sorbose, high fructose corn syrup, apple pulp, honey, sugar, maple syrup, pear juice, grape juice, orange juice, and fruit juice.

To facilitate delivery of a combiomic complex composition to a human or animal digestive tract, a combiomic complex composition may be combined with at least one member chosen from a group comprising capsule, sachet, pill, human food, pet food, pet treat, supplement, meat, dairy product, vegetable, fruit, fermented beverage, topical application, wound dressing, sunscreen, makeup product, mascara, cream, mouthwash, tampon, feminine pad, and dentifrice. According to one embodiment of the present teachings, a combiomic complex composition is delivered at a dose that provides at least about 1×10⁵ cfu per gram or about 1×10⁵ cfu per dose of bacteria and/or yeast included in the combiomic complex composition.

The teachings disclosed herein also provide a process for producing a combiomic composition. To this end, FIG. 2 shows certain salient steps of a combiomic complex producing process 200, according to one preferred embodiment of the present teachings. Process 200 begins with a step 202, which includes obtaining a pre-combiomic mixture. According to one embodiment of the present teachings, a pre-combiomic composition includes at least one bacteria and/or yeast, and at least one prebiotic. The different types of bacteria and/or yeast and prebiotics that may be used are discussed above. Also discussed above are other ingredients in a combiomic complex that may be first introduced in the pre-combiomic mixture.

Step 202 may include obtaining between about 1×10⁵ cfu of non-adhered bacteria and/or yeast per gram of non-adhered prebiotic and about 1×10¹⁰ cfu of non-adhered bacteria and/or yeast per gram of non-adhered prebiotic.

In other embodiments, the pre-combiomic mixture of the present teachings includes growth media and/or culture energy source. A growth medium is any fluid or gel that provides nutrients (e.g., vitamins and minerals) sufficient to promote the growth of one or more microorganisms. According to one embodiment of the present teachings, growth medium includes at least one member chosen from a group comprising beef broth, chicken broth, turkey broth, vegetable broth, lamb broth, pork broth, kangaroo broth, fish broth, salmon broth, meat broth, beef broth, chicken broth, seafood broth, beef, chicken, turkey, pork, lamb, kangaroo, fish, salmon, meat extract, beef extract, chicken extract, turkey extract, pork extract, lamb extract, kangaroo extract, fish extract, salmon extract, beef bouillon, chicken bouillon, turkey bouillon, pork bouillon, lamb bouillon, kangaroo bouillon, fish bouillon, salmon bouillon, dehydrated meat, dehydrated beef, dehydrated chicken, dehydrated turkey, dehydrated pork, dehydrated lamb, dehydrated kangaroo, Lactobacillus Selective (LBS) broth, Trypticase Soy Broth (TSB) supplemented with yeast extract and Tween 80, tryptic soy broth, APT broth, brain heart infusion broth, caseinates, calcium caseinate, sodium caseinate, potassium caseinate, hydrolyzed caseinate, corn steep solids, brewer's yeast, yeast extract, baker's yeast. In another embodiment of the present teachings, growth medium includes organ meat chosen from a group comprising heart, kidney, liver, brain, lung, thyroid, pancreas, spleen, and enteral.

A culture energy source or an energy source, i.e., a composition of fermentable carbohydrates in a liquid composition, serves a beneficial role of providing carbohydrate energy to aid the beginning stages of bacteria and/or yeast growth. An energy source may be thought of as part of a growth media, or it may be thought of as a substance that is added to a growth media. According to one embodiment of the present teachings, an energy source may include at least one member chosen from a group comprising apple juice, apple juice concentrate, dextrose, dextrose monohydrate, dextrose hydride, grape sugar, D-glucose, corn sugar, corn syrup solids, high fructose corn syrup, levulose, invert sugar, glucose, galactose, xylose, ribose, mannose, sorbose, high fructose corn syrup, apple pulp, honey, sugar, maple syrup, pear juice, grape juice, orange juice, and fruit juice. An energy source may also include a tea made from a fruit or vegetable. Examples include tea that is made from at least one member chosen from a group comprising chicory, pear, pineapple orange, apple, green bean, carrot, lentil, peas, and chick pea.

Next, a step 204 includes treating at least some of the pre-combiomic mixture to form a combiomic mixture, which includes at least one bacteria and/or yeast adhered to a prebiotic. In step 204, at least some of the non-adhered bacteria and/or yeast and at least some of the non-adhered prebiotic present in the pre-combiomic mixture are transformed and adhered bacteria and/or yeast and adhered prebiotic are formed. Although not necessary, this transformation may be facilitated by the secretion of a biofilm produced from the bacteria and/or yeast during step 204. According to certain embodiments of the present teachings, nearly all of the non-adhered prebiotic in the pre-combiomic mixture is transformed to adhered prebiotic in the combiomic mixture. Conversely, the combiomic mixture may include residual amounts of non-adhered bacteria and/or yeast.

To produce a combiomic mixture from a pre-combiomic mixture during treating step 204, bacteria and/or yeast may be inoculated in a growth medium. By way of example, a concentration of the growth medium used during inoculation is sufficient to generate at least about 1×10⁷ cfu/ml of bacteria in the combiomic mixture. In alternate embodiments of the present teachings, however, a bacteria and/or yeast is inoculated in a growth medium at a concentration that is sufficient to generate at least 1×10⁶ cfu/ml of bacteria in the combiomic mixture, at least 1×10⁸ cfu/ml of bacteria in the combiomic mixture, at least 1×10⁹ cfu/ml of bacteria in the combiomic mixture, at least 1×10¹⁰ cfu/ml of bacteria in the combiomic mixture, at least 1×10¹¹ cfu/ml of bacteria in the combiomic mixture, or at least 1×10¹² cfu/ml of bacteria in the combiomic mixture.

Treating of a pre-combiomic mixture in step 204 of FIG. 2 is preferably carried out at conditions sufficient to facilitate adhesion of the bacteria and/or yeast to the prebiotic component to produce a combiomic complex. In such manner, during step 204, a substantial population of non-adhered bacteria and/or yeast and non-adhered prebiotic inside a pre-combiomic mixture are transformed to adhered bacteria and/or yeast and adhered prebiotic, i.e., a combiomic complex, which along with the residual ingredients from a pre-combiomic mixture, comprise a combiomic mixture.

The specific treating conditions that facilitate formation of a combiomic complex in a combiomic mixture may vary widely. In one embodiment of the present teachings, step 204 includes conducting a temperature treatment, in which the temperature ranges from between about 25° C. to about 65° C., preferably between about 32° C. to about 49° C., and more preferably at about 37° C. The present teachings recognize, however, that any temperature value or values that facilitate adhesion of a prebiotic to a bacteria and/or yeast are acceptable.

Further, step 204 may be carried out for a duration that is between about 5 hours and about 48 hours, preferably between about 10 hours and about 16 hours, and more preferably, between about 12 hours and about 14 hours.

Further still, step 204 may be carried out at a pH value sufficient to facilitate growth and/or fermentation of a bacteria and/or yeast, as well as the resulting secretion of a biofilm by the bacteria and/or yeast. To this end, a pH value in a pre-combiomic mixture is preferably between about 5 and about 7.

In order to facilitate contact between a prebiotic and a bacteria and/or yeast during step 204, a pre-combiomic mixture may be periodically or continuously agitated.

In certain embodiments of the present teachings, step 204 is carried out under uniform conditions (e.g., time, temperature, pH) applied to a pre-combiomic mixture that includes at least one bacteria and/or yeast and at least one prebiotic. The pre-combiomic mixture subjected to uniform conditions may also include at least one energy source and at least one growth medium. In other embodiments of the present teachings, however, treating is carried out in multiple steps that may require separate treating conditions. The present teachings contemplate any combination of pre-combiomic mixture compositions and treatment conditions that facilitate adhesion of a bacteria and/or yeast to a secreted biofilm.

The present teachings also contemplate adding a combiomic complex to at least one member chosen from a group comprising sauce, condiment, probiotic drink, supplement, over-the-counter supplement, probiotic supplement, prepared food, ready-to-eat food, functional food, functional beverage, whole fruit, whole vegetable, kefir, butter, hummus, ketchup, mustard, and guacamole

Regardless of the specific conditions or ingredients used to transform a pre-combiomic mixture into a combiomic mixture according to the embodiment of FIG. 2, treating a pre-combiomic mixture, as described herein, may generate a combiomic mixture that includes a percentage value of an approximate number of adhered bacteria and/or yeast relative to an approximate total number of prebiotic that is between about 5% and about 100%, preferably at least about 35%, more preferably at least about 50%, and even more preferably at least about 75%, where percentage of adhered bacteria and/or yeast is calculated according to the expression:

X=A*100/(A+N)

where “X” is a percentage value of the amount of bacteria and/or yeast that adhere to a prebiotic, “A” is the approximate number of bacteria and/or yeast cells that adhere to a prebiotic, and “N” is the approximate number of bacteria and/or yeast cells that do not adhere to a prebiotic.

The number of adhered bacteria and/or yeast and the number non-adhered bacteria and/or yeast may be determined or counted by any method well known to those of skill in the art. According to certain embodiments of the present teachings, microscopy techniques (e.g., scanning electron microscopy, transmission electron microcopy, phase contrast microscopy, dark field microscopy, fluorescence microscopy, and confocal microscopy) are used to identify the number of adhered and non-adhered bacteria and/or yeast, preferably those that have been made visually perceptible using one or more stains. In another embodiment of the present teachings, light microscopy is used to determine the number of adhered and non-adhered bacteria and/or yeast. Similar techniques are used to determine the size of particles and microorganisms, and to identify sub-cellular structural components (e.g., capsular materials, biofilms, endospores, flagella, nuclei, mitochondria, endoplasmic membranes, and cell walls in eukaryotes of microorganisms). The microscopic field used to identify and characterize bacteria and/or yeast generally varies between about 500× and about 2000×.

Following step 204, a sample of the combiomic mixture may be, inter alia, delivered to a host for ingestion, preserved for long-term storage and later use, or temporarily stored at ambient temperature or in a refrigerator. According to one preferred embodiment of the present teachings, however, a combiomic complex is substantially separated from the liquid components of a combiomic mixture to produce a combiomic solid residue (which may also be used or preserved for later use). To this end, process 200 of FIG. 2 may further include a step of separating the combiomic complex from a combiomic mixture to form a combiomic solid residue that includes the combiomic complex.

Separating a combiomic complex from the other components of a combiomic mixture may be carried out by any method well known to those of skill in the art. According to one embodiment of the present teachings, separating is carried out by centrifuging the combiomic mixture at conditions sufficient to separate a combiomic complex from other components of a combiomic mixture. According to another embodiment of the present teachings, separating is carried out by sedimenting the combiomic mixture such that a substantial portion of the relatively heavier combiomic complex rests on the surface of a container that holds the combiomic mixture. According to yet another embodiment of the present teachings, separating is carried out by filtering the combiomic complex from the other components of the combiomic mixture. By way of example, filtering may be carried out through diatomaceous earth/silica sand. In yet another embodiment of the present teachings, separating is carried out by binding the combiomic complex to a flocculate or coagulant that can more easily be separated from the other components of a combiomic mixture. The present teachings recognize that any known method of separating, or any combination thereof, may be used to isolate a combiomic complex.

According to one embodiment of the present teachings, separating the combiomic complex from the combiomic mixture produces a combiomic solid residue with a moisture content. The combiomic solid residue is at most about 20% by weight of the combiomic mixture, more preferably at most about 10% by weight of the combiomic mixture, even more preferably at most about 5% by weight of the combiomic mixture, and most preferably less than about 5% by weight of the combiomic mixture. Thus, the present teachings disclose processes that produce relatively concentrated doses of a combiomic complex or solid residue.

Providing a concentrated dose of a combiomic complex to a human or animal host provides certain advantages. A concentrated dose of a combiomic complex delivered to the oral cavity or gut tends to be more efficacious and faster acting in promoting health than delivering a relatively less concentrated dose. Further, delivering a more concentrated dose counterbalances any loss or degradation that may occur to the bacteria and/or yeast, or combination thereof, when passing through a host's intestinal environment. Finally, when stored and/or preserved, a combiomic complex provides more stable bacteria in the combiomic complex, because the separating step selects for bacteria that are more viable. The present teachings recognize that bacteria in a combiomic complex produced according to the present teachings are relatively more stable, as these bacteria are non-planktonic, i.e., adhered. In other words, because non-planktonic bacteria are more competitive in establishing a niche within an environment by attaching to a surface, bacteria associated with a combiomic complex may generally be thought of as stronger, or more desirable, than their planktonic counterparts.

A combiomic solid residue may be ingested fresh (e.g., in a capsule, pill or suppository), applied topically (e.g., in a cream, spray or lotion), added to food, or stored for extended lengths of time. By way of example, a combiomic solid residue may be added to pet food, human food (e.g., ice cream, yogurt, milk, meat, or fermented meat). The combiomic may be dried to a powder and ingested as a pill. The combiomic may also be added to bandages, suppositories, mouth wash, dentifrice, confection, desert, eye drop, ear drop, vaginal cream, or be encapsulated for ingestible applications, incorporated into chewing gum, or applied to cotton or rayon-tipped swabs.

FIG. 3 shows certain salient steps of a process for promoting human or animal health 300, according to one embodiment of the present teachings and that uses a combiomic complex. Process 300 begins with a step 302, which includes providing a combiomic complex. Providing a combiomic complex may include providing a combiomic complex that is contained in at least one carrier chosen from a group comprising capsule, sachet, pill, human food, pet food, pet treat, supplement, meat, dairy product, vegetable, fruit, beverage, and fermented beverage. According to another embodiment of the present teachings, providing includes providing a combiomic complex that is prepared in a topical application that is at least one member chosen from a group comprising spray, wound dressing, sunscreen, makeup product, mascara, cream, mouthwash, tampon, feminine pad, and dentifrice.

In one embodiment, step 302 of the present teachings includes obtaining a pre-combiomic mixture (e.g., step 202 of FIG. 2) and/or treating the pre-combiomic mixture to form a combiomic mixture (e.g., step 204 of FIG. 2). Various embodiments of these above-described steps are also applicable to implement this embodiment of step 302.

Next, a step 304 includes directing use of the combiomic complex by a human or animal. Directing use may include instructing, prescribing and advising. According to one embodiment of the present teachings, directing use of a combiomic complex includes directing use of between about 1×10⁵ cfu and about 1×10¹⁰ cfu of bacteria and/or yeast in the combiomic complex per gram of combiomic complex. Directing use may be thought of as facilitating ingestion of the combiomic complex by a human or animal. Alternatively, directing use of a combiomic complex may include topical application of the combiomic complex on a surface of a human or animal. According to one embodiment of the present teachings, a surface of a human or animal is at least one member chosen from a group comprising tooth, gum, skin, mucous membrane, and ear canal.

A combiomic complex may be used for treating any number of health conditions. According to one embodiment of the present teachings, treating a health condition includes treating at least one member chosen from a group comprising heart disease, dental carry, intestinal disorder, irritable bowel syndrome, diarrhea, acne, skin wrinkles, skin disease, gum disease, plaque formation, gum infection, inflammation, and improving healing during tooth removal. In this context, step 304 may provide directions on amount and/or frequency of doses of the compound that includes the combiomic mixture to treat such diseases.

A combiomic complex and associated processes disclosed herein are particularly useful for treating health conditions due to the presence of beneficial or health-promoting bacteria in a combiomic complex. The present teachings recognize that beneficial bacteria and/or yeast adhered to a combiomic complex is more resistant to acid exposure (e.g., acids found in the digestive system of a human or animal) than non-adhered bacteria and/or yeast. According to one embodiment of the present teachings, a biofilm associated with a combiomic complex facilitates adhesion of beneficial bacteria to a surface of an intestine of a human or animal host after consumption of the combiomic complex. Preferably, the biofilm includes one or more acid mucins that contain similar binding sites for the bacteria as the surface villi of the intestine of the human or animal. As such, the biofilm in the combiomic complex provides bacteria that are capable of binding to the surface villi of the intestine of a human or animal, facilitating delivery of beneficial bacteria to the host. According to yet another embodiment of the present teachings, consumption of a combiomic complex facilitates development of microbiota that promotes human or animal health in at least one member chosen from a group comprising mouth, intestine, and colon.

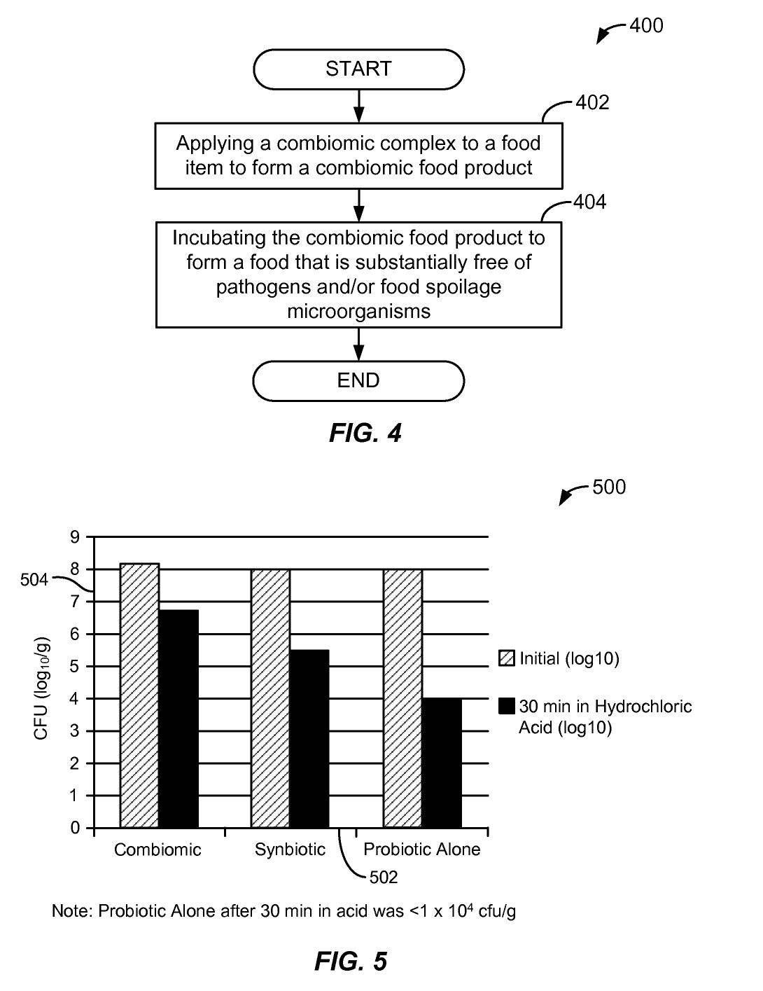

FIG. 4 shows certain salient steps of a process 400 for promoting pathogen control and/or safety of food, according to one preferred embodiment of the present teachings. Process 400 begins with a step 402, which includes obtaining a combiomic complex and a food. A food may be at least one member chosen from a group comprising ice cream, yogurt, milk, meat, fermented meat, kibbled food, kibble, expanded food, pelleted food, extruded food, refrigerated food, refrigerated treat, frozen food, frozen treat, biscuit, raw food, fried food or treat, soft-moist food or treat, pellet, fine, broken piece of food, jerky-style treat, injection-molded treat, treat, supplement, prepared salad ingredient, ground fruit or vegetable, whole fruit, whole vegetable, prepared meal, slaughtered carcass, prepared food, meat piece or chunk, fabricated meat chunk, fabricated protein chunk, livestock feed, steam-flaked feed, and aquaculture feed. In one embodiment, step 402 is substantially similar to step 302 of FIG. 3. Step 402, similar to step 302, includes obtaining a pre-combiomic mixture (e.g., step 202 of FIG. 2) and/or treating the pre-combiomic mixture to form a combiomic mixture (e.g., step 204 of FIG. 2). Various embodiments of these above-described steps are also applicable to implement this embodiment of step 402.

Next, a step 404 includes applying to the food a combiomic complex to form a combiomic food product. Preferably, applying the combiomic complex to the food produces a combiomic food product that has between about 1×10⁵ cfu of bacteria and/or yeast per gram of the combiomic food product and about 1×10¹¹ cfu of bacteria and/or yeast per gram of the combiomic food product.

Then, a step 406 is carried out. Step 406 includes incubating the combiomic food product to form a food product that is substantially pathogen free. Incubating may include a suitable temperature and other treatment conditions sufficient to produce a substantially pathogen free food product. According to one embodiment of the present teachings, a substantially pathogen free food has less than about 100 cfu of pathogen per gram of the food product.

According to another embodiment of the present teachings, a method for promoting food preservation is disclosed. Using steps that are substantially similar to those disclosed with respect to process 400 of FIG. 4, a shelf-stable food may be produced. According to one embodiment of the present teachings, producing a shelf-stable food includes using a food-preservation bacteria in a combiomic complex.

According to one embodiment of the present teachings, a combiomic complex in a combiomic food product includes at least one bacteria that facilitates pathogen control and/or at least one bacteria that promotes food preservation on a combiomic food product. According to another embodiment of the present teachings, a combiomic complex also includes at least one bacteria that promotes human or animal health. According to this embodiment, the presence of a pathogen-control bacteria and/or a food-preservation bacteria with a health-promoting bacteria on or in a food product facilitates safety and/or preservation of the food product prior to consumption by a human or animal, and then promotes health of a human or animal after consumption of the food product.

The systems, methods, and compositions disclosed herein contemplate any number of uses of a combiomic complex in addressing human or animal health or food preservation and/or safety. A combiomic may be used to aid in the development of favorable oral, intestinal and/or cecal/colonic microbiota when ingested by a human or an animal. By way of example, a combiomic complex may deliver bacteria to the colon to address diarrhea or other intestinal conditions. The present teachings recognize that adhesion of bacteria and/or yeast to a prebiotic creates a healthier intestinal environment by competing for attachment at the villi. In the above example, the combiomic complex contains a bacteria and/or yeast that induces biofilm formation made up of acid mucins that are similar to the acid mucins associated with the intestinal wall, thus providing an advantage to the bacteria and/or yeast associated with the combiomic to adhere to the intestinal wall. The result of this competitive adherence is a lower likelihood of pathogenic bacteria to attach to the villi of the intestine. Decreased pathogenic bacteria attachment results in less intestinal diseases. In certain of such embodiments, a combiomic complex is delivered in a mouthwash contained in a sachet.

The present teachings recognize that a combiomic may be used to treat any disease or health condition associated with growth and/or metabolism of bacteria in a host. Examples include diseases of the gastrointestinal tract (including the esophagus, stomach, small intestine, large intestine and rectum), gastrointestinal inflammation, gastrointestinal ailment, skin condition, skin inflammation, excess gas formation, bloating, indigestion, diarrhea, acid reflux/heartburn, irritable bowel syndrome, inflammatory bowel disease, GERD, gastroesophageal reflux disease, Crohn's Disease, diverticulitis, ulcerative colitis, celiac disease, psoriasis, eczema, Grave's disease, autoimmune disorders, constipation, hemorrhoids, anal fissures, diverticular disease, polyps, colitis, infectious colitis, ulcerative colitis, and ischemic colitis.

As but one example, a combiomic complex may deliver bacteria to the oral cavity to outcompete deleterious plaque formation on teeth and gums. The present teachings recognize that dental gums frequently have biofilms created by pathogenic bacteria. These biofilms result in plaque formation and ultimately tartar buildup. An additional benefit of delivering active bacteria and/or yeast to various endogenous sites (e.g., dental gums or intestinal villi) is that such bacteria and/or yeast create a beneficial (rather than detrimental) biofilm. Bacteria and/or yeast attached to a prebiotic create an environment where the bacteria and/or yeast out-compete the pathogenic bacteria and the above-mentioned pathogenic biofilm is not created. In fact, the beneficial bacteria and/or yeast maintain healthier gums. In an alternate embodiment of the present teachings, a combiomic complex is used as a dental health supplement in human health for treating any dental condition implicating bacterial or pathogenic infection. In this context, step 304 of FIG. 3 includes providing directions on amount of doses and frequency of doses of the compound, which includes the combiomic mixture, to curtail or cure such dental conditions.

In one embodiment of the present teachings, a combiomic complex is included in a digestive health supplement for humans. Though various combiomic amounts and delivery mechanisms are contemplated by the present teachings, one preferred embodiment of the present teachings includes delivering a combiomic comprising Pediococcus acidilactici and Pediococcus pentosaceus attached to chicory. By way of example, approximately 425 mg of a combiomic complex (sufficient to provide about 9×10⁸ cfu/dose of supplement) may be delivered via a sachet that also includes mouthwash. Other delivery mechanisms for the same composition include delivery in a capsule, adding to a food product (e.g., meat), mixing with a saline mouthwash.

In another embodiment of the present teachings a combiomic complex is included in a digestive health supplement for pets. Though various combiomic amounts and delivery mechanisms are contemplated by the present teachings, one preferred embodiment of the present teachings includes delivering a combiomic comprising Pediococcus acidilactici and Pediococcus pentosaceus attached to chicory, in the presence of a flavor and/or palatability enhancer. By way of example, approximately 425 mg of a combiomic complex (sufficient to provide 9×10⁸ cfu/dose supplement) and approximately 200 mg of flavor and/or palatability enhancer may be delivered via a sachet that is applied to a pet's meal before consumed. In certain embodiments of the present teachings, a combiomic complex is added to a pet's food prior to consumption. Alternately, a combiomic complex is included in a solution delivered using a sachet.

In yet another embodiment of the present teachings, a combiomic complex is included in the composition of a dental health biscuit for animals. As but one example of such a biscuit, a combiomic complex that includes Pediococcus acidilactici and Pediococcus pentosaceus (preferably sufficient to provide about 4.2×10⁸ cfu/biscuit) attached to chicory is used. The biscuit may be comprised of the following ingredients, by percentage of formula: wheat flour (26.7%); ground corn (13.4%); rolled oats (13.4%); water (11.3%); butter (8.0%); wheat germ (5.3%); parsley (5.3%); mint leaves (5.3%); chicken fat (4.3%); fish oil (2.1%); baking powder (0.7%); salt (0.3%); combiomic complex (2.1%). The combiomic complex is preferably added after the biscuit is baked to minimize heat deactivation of the bacteria. Using this recipe, two biscuits per day are preferably fed to an animal to facilitate improved dental health.

In yet another embodiment of the present teachings, a combiomic complex is added to a beverage. Beverages may include those containing whole fruits, vegetables and any be made from any suitable fruit or vegetable such as, but not limited to, carrot, cranberry, orange, blueberry, tomato, apple, lemon, lime, grape, strawberry, grapefruit, tangerine, mandarin orange, tangelo, pomelo, celery, beet, lettuce, spinach, cabbage, artichoke, broccoli, brussel sprouts, cauliflower, watercress, peas, beans, lentils, asparagus, onions, leeks, kohlrabi, radish, turnip, rutabaga, rhubarb, carrot, cucumber, zucchini, eggplant, pineapple, peach, banana, pear, guava, apricot, watermelon, lingonberry, blueberry, plains berry, prairie berry, mulberry, elderberry, choke cherry, date, coconut, olive, raspberry, strawberry, huckleberry, loganberry, currant, dewberry, boysenberry, kiwi, cherry, blackberry, quince, buckthorn, passion fruit, rowan, gooseberry, pomegranate, persimmon, mango, papaya, lychee, plum, prune, and figs. Beverages may also include tea, coffee, carbonated drink, refreshing drink, sport drink, meal replacement beverage, fortified beverage, electrolyte containing beverage, nutritional supplement beverage, performance drink, energy drink, and the like.

In one embodiment of the present teachings, one or more bacteria and/or yeast are attached to a fruit or vegetable pulp or puree (e.g., apples, pears, bananas, oranges, tomato, peas, green beans, carrots, and squash). According to the present teachings, the pulp or puree provides a sufficient energy source comprising free monosaccharides, disaccharides, and oligosaccharides (i.e., fiber) for growth of a microorganism and its adherence to an oligosaccharide of the fruit or vegetable, which serves the function of a prebiotic. Accordingly, no additional prebiotic source would be needed. In one such embodiment of the present teachings, a mixture comprising: (i) fruit or vegetable pulp or puree in the amount of about 99.9474% by weight of the mixture; and (ii) starter culture in the amount of 0.0626% by weight of the mixture, is used.

In another embodiment of the present teachings, a cheese or curd is made from a nut milk (e.g., almond milk) or soy beans via adhesion to bacteria and/or yeast. According to this embodiment, a bacteria and/or yeast is introduced, which ferments the nut milk or the soy beans to generate a curd. In doing so, the bacteria and/or yeast will adhere to the nut milk or soy bean curd, which serves as a prebiotic, thus producing a combiomic complex. Thus, according to the present teachings, a non-dairy food product is produced to provide beneficial bacterial effects.

In yet another preferred embodiment of the present teachings, a combiomic complex includes one or more bacteria that have attached thereto one or more bacterial microcompartments, i.e., bacterial organelles that are made of a protein shell that surrounds and encloses various enzymes. By way of an example of the present embodiment, a bacterial microcompartment that is capable of making a tumor necrosis factor-related apoptosis inducing ligand (TRAIL), a drug that is used to treat cancer cell growth, may be attached to a bacteria and/or yeast that is used in a combiomic complex. Such a combiomic complex may be administered orally (e.g., in a pill form). According to the present teachings, a combiomic complex is capable of surviving the acid environment of the stomach and delivered to the large intestine where a pre-cancerous polyp may be located. Further, the combiomic provides an energy source for the bacterial microcompartment to generate TRAIL that then inhibits the growth of the pre-cancerous polyp.

The present teachings recognize that bacterial microcompartment may be attached to a bacteria and/or yeast. By way of example, Shewanella bacteria and L. acidophilis may be cultured together, harvested, and any population of L. acidophilis that incorporated the bacterial microcompartments of the Shewanella bacterial may be isolated for further use. As another example, attaching a bacterial microcompartment to a bacteria may include the following steps: (i) fragmenting a lactic acid bacteria, a Bacillus coagulans, or Shewanella by sonication; (ii) recovering the resulting fragments (e.g., by ultracentrifugation); and (iii) attaching the recovered fragments that contain bacterial microcompartments to proteins from the target bacteria. The bacterial microcompartments form ligands through the binding of amino acid reactive groups between the bacterial microcompartments and the host bacteria. Examples of target bacteria may include L. paracusei, L. Plantarum, L. Rhamnosus, L. fermentum, and L. Salivarius.

In still yet another preferred embodiment of the present teachings, a combiomic complex may be used in dental health application to inhibit activity of Streptococcus mutans, which is known to cause dental carries. According to the present embodiment, a combiomic complex may effectively deliver bacteria to address dental carries. By way of example, a bacteria and/or yeast may be adhered to prebiotics that are known to be “sticky” or gummy. In particular, prebiotics, which exist in the form of viscous fibers, such as B-glucan, Carboxymethylcellulose (CMC), Methylcellulose, Hydroxypropylmethylcellulose (HPMC), Psyllium, Guar gum, Citrus pectin, Pectin, Xanthan gum, Gum Arabic, Gum talha, and Alginate may be used. In another embodiment, a combination of prebiotics, e.g., CMC and inulin/FOS, are adhered to a bacteria and/or yeast. According to the present teachings, a combiomic complex is able to outcompete S. mutans due to the readily available food source (i.e., prebiotic). The use of a viscous fiber, i.e., prebiotic, in such a complex facilitates adhering of the complex, which includes the bacteria and/or yeast, to the oral cavity. As another example, use of mucopolysaccharides (e.g., xanthan gum) to bind the combiomic complex to teeth/gums, along with the combination of prebiotics to fuel the probiotics (present in the form of bacteria and/or yeast) that are attached to it, may be useful in addressing health concerns associated with dental carries. Further applications include, but are not limited to, treating receding gum disease, decreasing plaque formation, decreasing gum infections due to insults on the gums, and improved healing during tooth removal.

In yet another embodiment of the present teachings, one or more combiomic complexes present a natural and compatible solution to the problem of adding probiotics (present in the form of bacteria and/or yeast) to many foods and orally ingestible medicinals. By way of example, combiomic complexes are compatible with fat-enriched foods and medicines. The fat provides additional stability to aerobically sensitive bacteria strains, while the attachment to a prebiotic provides greater stability within the intestinal tract for passage of the combiomic complex to an active site in the lower intestine.

In yet another embodiment of the present teachings, a combiomic complex is used to deliver, to the intestinal tract, bacteria that facilitate the production of short-chain fatty acids (e.g., butyrate), which are useful, among other reasons, in treating cancer.

In yet another preferred embodiment of the present teachings, topical applications of combiomic complexes provide energy sources for actions of live bacteria. Specific applications include bandages impregnated with a combiomic complex; delivering bacteria that control odor in deodorants, sunscreen, acne treatments, wrinkle treatment and prevention, and treating various skin diseases and conditions (e.g., psoriasis). Yet another application could include: a nose spray to treat the sequelle (i.e., secondary infection like Shingles or Scarlett Fever), Staphylococcus aureus sinus infections form a viral rhinitis; and the common cold. In particular, topical applications with low water activity/moisture level may be useful. Examples of topical applications include, but are not limited to, emollient, shea butter, lanolin, cocoa butter, butter crèmes, paw paw, and cosmetics. Likewise, topical applications that incorporate fruit purees may be used. Bacteria and/or yeast would attach to the fibers in these fruits such that an additional source of fiber (prebiotic) would not be necessary. Further, cream-based products provide stability to the probiotic (present in the form of bacteria and/or yeast) delivered topically, as the water levels in such application would be low. In another embodiment of the present teachings, a combiomic may be used to treat throat infections (e.g., Streptococcoccus pyogenes).

In yet another embodiment of the present teachings, a combiomic is coated onto the surface of pet food kibbles. The combiomic provides the advantages of providing additional resistance, to a bacteria, from surface acids associated with palatant digests, which range from a pH of about 3 to a pH of about 5, that are applied to the kibbles. In other words, the prebiotic that is attached to the bacteria and/or yeast, or combination thereof, enhances the efficacy of a live bacteria and/or yeast, or combination thereof, that is delivered to an animal consuming a pet food kibble, thus providing the animal one or more benefits associated with the bacteria and/or yeast, or combination thereof, after the pet food kibbles have been consumed.

In yet another embodiment of the present teachings, a combiomic complex is added to a raw chicken meat (e.g., emulsified organs, muscle, cartilage and bones) prior to its inclusion as an ingredient into a pet food. The combiomic complex, according to the present teachings, increases the shelf-life of the raw meat ingredient. Further, a combiomic complex provides the advantage of faster growth over adding only a shelf-stability enhancing bacteria to the raw meat due to the presence of a food supply (i.e., prebiotic). Similarly, a combiomic complex added to raw meat also provides the advantage of increased efficacy of the bacteria in the gastrointestinal tract due to the presence of a food supply (i.e., prebiotic).

In yet another embodiment of the present teachings, a combiomic is added to a cooked pet food to stabilize the food for up to 24 months. In such embodiments, the combiomic complex provides the advantage of a faster growth rate of the combiomic probiotic due to the presence of a prebiotic in the combiomic complex, and enhanced gastrointestinal benefits when the bacteria and/or yeast in the combiomic complex is delivered to the digestive system.

In yet another embodiment of the present teachings, a combiomic complex is delivered, via a liquid (e.g., as a spray) to the surface of at least one member chosen from a group comprising fruit, vegetable, meat, seafood, and pet food. According to the present teachings, applying a combiomic complex to the surface of a food does not require that the food be cooked. Accordingly, a combiomic complex may be applied to a food while it is in its native state (e.g., a fruit or vegetable prior to harvesting), after a food is harvested, during manufacturing of a food product, or during or after packaging. In such embodiments, a bacteria and/or yeast, or combination thereof, provides several advantages, including delivery of a bacteria to various parts of the body after ingestion, enhanced food safety and protection against pathogens, extended shelf-life in the supply chain (and therefore reduced waste), improved taste/palatability, and simpler and more natural ingredient lists for consumer appeal, which is consistent with current consumer trends.

In yet another embodiment of the present teachings, a combiomic complex is used to deliver a probiotic (which is in the form of bacteria and/or yeast) in raw meats and fish in slaughterhouses, fisheries, and abattoirs. According to such embodiments, to keep combiomic inoculation costs low in slaughterhouses or fisheries, a combiomic culture is subcultured (e.g., 400 cultured batches may be used to inoculate 40,000 batches) so that a new culture does not need to be used, facilitating combiomic colonization of meat or fish. In addition to delivering the combiomic complex to the host's gastrointestinal tract to provide certain health benefits, a combiomic complex will also provide the advantage of protecting a host animal or human from pathogens attached to the food, thus facilitating food safety and protection against spoilage organisms, thus providing preservation. An additional benefit of applying a combiomic complex to raw meat or fish includes extending shelf life of a raw meat or fish and enhancing palatability and texture of the raw meat or fish for later consumptions.

In yet another embodiment of the present teachings, a combiomic complex is used to treat raw meats and fish in slaughterhouses, fisheries, and abattoirs. According to such embodiments, raw meat or fish is coated with a mixture that includes a combiomic complex, facilitating inhibition of pathogen and/or spoilage microorganism growth on raw meat or fish. The present embodiment provides the further advantage of creating a more sterile environment for further processing and for employees. Further, the present embodiment results in an extended shelf-life and less color loss that may otherwise occur due to a meat or fish being in a raw state. Finally, meat and fish coated with a combiomic complex provides the additional health benefit of delivering a combiomic complex to the gastrointestinal tract after the meat or fish is ingested. According to the present teachings, such health benefits are in part realized because a combiomic complex facilitates survival of a probiotic during delivery through the digestive tract and provides an energy source (i.e., a prebiotic) for the probiotic inside the digestive tract.

In yet another embodiment of the present teachings, a combiomic complex is sprayed onto the surface of agricultural products, including eggs, to reduce the risk of salmonella and pathogen transfer to humans. Such an embodiment provides the benefit of an extended shelf-life, a reduction of waste, and higher agricultural yields.

In yet another embodiment of the present teachings, a combiomic complex is sprayed onto the surface of pet food ingredients, including rendered meats, meat by-products, fish meals, meat and bone meals, meat meals to reduce the risk of salmonella and pathogen transfer into pet food manufacturing or processing plants. Such an embodiment provides the benefit of reduced risk of pathogen load in pet food manufacturing or processing equipment, minimizes the risk of catastrophic extrusion failure, and provides a lower risk operating environment for pet food manufacturing or processing employees or other individuals that may come in contact with rendered meats, meat by-products, fish meals, meat and bone meals, and meat meals.

In yet another embodiment, a combiomic complex of the present teachings is sprayed or mixed into animal feed. According to this embodiment, such spraying or mixing provides the advantage of reducing the pathogenic bacteria load on poultry, pork, fish farms, or cattle yards, which in turn reduces the pathogenic load of bacteria in the final meats. The present embodiment provides the further advantage of delivering a combiomic complex to the gastrointestinal tract of the ingesting animal, which will facilitate health of that animal.

In yet another embodiment, a combiomic complex of the present teachings is sprayed or mixed into animal feed. According to this embodiment, such spraying or mixing provides the advantage of reducing the pathogenic bacteria load on poultry, pork, fish farms, or cattle yards, which in turn reduces the pathogenic load of bacteria in the final meats. The present embodiment provides the further advantage of delivering a combiomic complex to the gastrointestinal tract of the ingesting animal, which will facilitate health of that animal.

In yet another embodiment of the present teachings, a combiomic complex is sprayed onto or mixed in food in a food service area, a food display area, or a salad bar, to prevent pathogen activity and to ensure food safety.

In yet another embodiment of the present teachings, a combiomic complex is mixed into or used as a surface coating for at least one member chosen from a group comprising bread, snack, cereal, semi-moist pet food, soft moist pet food, wet pet food, dry pet food, and pet treat.

The present embodiment provides several advantages, including delivery of a bacteria and/or yeast and a prebiotic to a consumer's gastrointestinal tract, prevention of pathogen ingestion, food safety assurance, enhanced palatability of food, and simpler and more natural ingredient lists.

In yet another embodiment of the present teachings, a combiomic complex is added to at least one member chosen from a group comprising confection, candy, raw jam or jelly, cooked jam or jelly, preserve, spread, dip, and peanut butter.

In yet another embodiment of the present teachings, a combiomic complex is placed in a sweet and sour supplement that is in a shelf-stable form for refrigeration. Such a supplement may be spooned into meals and desserts in a variety of forms. Such an embodiment provides the advantages of enhanced taste/palatability, better nutrition, simpler and more natural and healthy ingredient lists for consumer appeal, and extended shelf life of the products.

In yet another embodiment of the present teachings, a combiomic complex is added daily to foods, meals, supplements, and pharmaceuticals. According to the present teachings, a combiomic complex may be added using a sweet or savory application that is prepared in the form of a liquid, gel, powder, or other additive that is applied directly into a food, a gellified product that can be spread onto a food, and a food that may be consumed directly (e.g., a chocolate flavored combiomic complex).

In yet another embodiment of the present teachings, a combiomic is applied to a pet product to reduce pathogenic activity in a home environment. By way of example, a pet product may include, without limitation, a pet pad or a pet litter.

In yet another embodiment of the present teachings, a combiomic is obtained by subculturing a larger culture that includes a combiomic complex. By way of example, a kettle may be fitted with a thermal heating jacket that is capable of holding about 100 kg of culture. 97.9374 kg of chicken broth is added into a kettle and heated to 105° F. 1 kg of dextrose (culture energy source), 1 kg of ground chicory (prebiotic) and 0.0626 kg of Pediococcus acidilactici and Pediococcus pentosaceus (starter culture, at a level sufficient to provide 1×10⁷ cfu/g of solution) are additionally added to the heated chicken broth. The mixture is incubated for 24 hours. At the end of 24 hours, 50 kg of the incubated mixture is removed and centrifuged at 4,000 rpm for 30 min to remove the liquid supernatant portion, and the solid residue is collected. The solid residue is mixed, for example, in a mixing tank, with glycerol and citric acid to form a new combiomic mixture in the following proportions: solid residue (i.e., combiomic complex) in the amount of 60% by weight of the new combiomic mixture, glycerol in the amount of 30% by weight of the new combiomic mixture, citric acid in the amount of 10% by weight of the new combiomic mixture. This new combiomic mixture is stored in a storage space at −10° F. until further use. The remaining 50 kg of the incubated culture is used as a starter culture in another or same kettle for making a subsequent 100 kg incubated batch. To make the subsequent incubated batch, the following are added into the remaining 50 kg batch: 49 kg of chicken broth, 0.5 kg of dextrose, and 0.5 kg of chicory. The mixture is heated at 105° F. for 6 h or until to the pH is less than 4.7. After incubation, 50 kg of the subsequent incubated batch is removed, and a solid residue that includes the combiomic complex is separated as described above, and the process of obtaining another 100 kg batch is repeated. Subculturing a combiomic complex, according to the present teachings, provides the advantages of maintaining a continual process of culturing, reducing the costs of an inoculum, and increasing the speed at which a combiomic complex is generated.

In yet another embodiment of the present teachings, a combiomic is coated onto a pet food product. After kibbles are extruded, they subsequently undergo drying to a shelf-stable (less than 12%) moisture level. Separately, a combiomic complex is mixed into a dry palatant at about 0.1% to about 10% by weight of the palatant to produce a combiomic-enriched palatant that contains between about 1×10⁵ and about 1×10¹⁰ cfu of combiomic bacteria per gram of combiomic-enriched palatant. After drying, the kibbles are gravity dropped past an APEC disc coater and coated with a blend of fat- and combiomic-enriched palatant to between about 1% and about 15% of the final weight of the product to produce fat-coated kibbles. Further blending of the fat and kibbles continues in a horizontal ribbon mixer. As a result of coating the kibbles with the combiomic-enriched palatant in the APEC disc coater and subsequent horizontal ribbon mixer, the resulting combiomic-containing kibbles have between about 1×10³ and about 1×10⁹ cfu of the combiomic bacteria per gram of the combiomic-containing kibbles.

In yet another embodiment of the present teachings, an apparatus that facilitates production of a combiomic is disclosed. By way of example, a stainless steel kettle fitted with a thermal heating jacket may be used as a basin in which mixing and incubation of a synbiotic or pre-combiomic mixture, to produce a combiomic mixture, is carried out. A thermal heating jacket is capable of heating the apparatus up to 130° F. and maintaining this temperature in ambient environments as low as −20° F. The kettle may include within it a vertically oriented mixing shaft that is oriented in an auger formation. The vertical oriented mixing shaft is capable of rotating on the vertical axis at a rate of up to 30 rotations per minute. The kettle may further be designed to be closed to prevent outside air from continuously contaminating the kettle's contents. Further, the kettle may maintain a one-way gas release valve to prevent internal contents of the kettle from pressurizing. Further, the kettle may include a thermo-coupled probe to monitor temperature. The kettle may further include a probe designed to monitor pH of the solution contained within the kettle. The kettle may further include a port opening on top of the kettle through which contents of the kettle may be added, and/or a port opening on the bottom of the kettle through which contents of the kettle may be removed. Once removed, contents that include the combiomic mixture may be conveyed to one or more other apparatuses for further processing. By way of example, the one or more apparatuses may be used to separate a combiomic solid residue (e.g., a separating tank), dry the combiomic mixture (e.g., a drying tank), and/or prepare the combiomic mixture and/or combiomic complex for conveyance to a host (e.g., as part of a pill, capsule, suppository, or the like). As another example, the kettle or any other apparatus that processes a combiomic may be fitted with a means of delivering the combiomic to a food-inoculating tank where the combiomic is added to a food product (e.g., to promote food safety or food preservation, and to serve as vehicle for delivering beneficial bacteria to a host that eats the food).

In other embodiments of the present teachings, a combiomic complex is used to coat the surface of a food product. In such embodiments, a combiomic complex may be used to coat surfaces in a mixture that includes a solid combiomic complex in a liquid (or a combiomic mixture). The term “original combiomic complex fluid” is used to describe the fluid source of which contains the combiomic solids. “Concentrated combiomic fluid” is the term used to describe the fluid that contains an increased amount of combiomic solids due to any means of concentrating the combiomic solids from the original combiomic complex fluid. The amount that the concentrated combiomic fluid has been concentrated may be expressed by the following ratio: number of volumes of the original combiomic complex fluid to one volume of concentrated combiomic fluid. Various degrees of concentration of the original combiomic complex fluid may be obtained. The present teachings recognize that the ratio of number of volumes of the original combiomic complex fluid to one volume of concentrated combiomic fluid may be a value that is about 1:1, about 2:1, about 4:1, or about 8:1. The present teachings recognize that using a combiomic complex, at relatively higher concentrations, to coat the surface of foods provides additional “killing power” to reduce the pathogen activity on the surface of a food. According to preferred embodiments of the present teachings, a combiomic complex has a concentration of about 8:1 by weight of the combiomic mixture when applied to the surface of a food.

### EXAMPLES

### Example 1

### Preference of a Non-Adhered Bacteria that is Adhered to a Prebiotic

In the embodiment of example 1, a bacteria's preference for adhering to a prebiotic over remaining a non-adhered bacteria during a treating step (e.g., treating step 204 of FIG. 2) is experientially shown. To this end, Table 1 shows a composition of a pre-combiomic mixture used to generate samples to test the affinity of a bacteria for adhesion to a prebiotic.

TABLE 1
Composition Used to Create “Pre-Combiomic Mixture”

| Ingredient | Percent by Weight |
|---|---|
| Beef broth | To 100 |
| Apple juice concentrate | 1* |
| Chicory | 1 |
| Starter Culture** | 0.0626 |

*Starter Culture contains sufficient Pediococcus acidilactici and Pediococcus pentosaceus (i.e., non-adhered bacteria) to provide 1 × 10⁷ cfu/ml of beef broth (i.e., growth medium). The concentration, 0.0626%, is an approximate concentration. As the concentration of bacteria in a starter culture may vary, the actual percentage included in the Fermented Mixture may vary.
**Apple juice concentrate (i.e., culture energy source) was added to the beef broth to provide monosaccharides at about 1% of the composition. As a result, apple juice concentrate was added at 36%, by weight, of the pre-combiomic mixture to achieve the addition of 1% monosaccharides from apple juice concentrate.

Using the recipe shown in Table 1, apple juice concentrate was added to the beef broth to achieve about a 1% solution, by weight, of monosaccharides in solution. Then, Pediococcus acidilactici and Pediococcus pentosaceus were inoculated into the broth to achieve about 1×10⁷ cfu/ml of solution. Chicory (i.e., prebiotic) was also added to the inoculated beef broth and apple juice concentrate to achieve about a 1% solution, by weight.

This mixture was then treated for about 24 hours at about 45° C. After decanting a liquid supernatant, a combiomic solid residue (which includes a combiomic complex) was used as the source of samples that provided the results shown in Tables 2 and 3. In order to determine the formation of a combiomic complex, two visual verification techniques were performed according to techniques well-known to those of skill in the art.

First, using a “wet stain” technique, a sample of the combiomic solid residue was placed on a slide and stained with toluidine blue 0. The slide was rinsed, a drop of immersion oil was added onto a cover slip, and the slide was examined under a light microscope (AmScope Compound) at 1000 to 2000 times magnification. Table 2 shows the results after counting the formation of combiomic complexes on four randomly obtained slide samples. As shown in Table 2, 86.8% of bacterial cells were adhered to chicory; only 13.2% of the bacterial cells were not adhered to the chicory.

TABLE 2
Results after Evaluating Pediococci under Light
Microscopy Using a Wet-Stain Technique

| | | Total Number | Total Number |
|---|---|---|---|
| Obser- | Total Number | of Pediococci | of Pediococci Not |
| vation | of Pediococci | Attached to Chicory | Attached to Chicory |
| 1 | 0 | 0 | 0 |
| 2 | 69 | 49 | 20 |
| 3 | 0 | 0 | 0 |
| 4 | 90 | 89 | 1 |
| Average | 159 | 138 (86.8%) | 21 (13.2%) |

Second, a “dry stain” technique was used. A sample of the combiomic solid residue was placed on a slide, dried (without heat), fixed to the slide using mild heat from a heat source, stained (with Crystal Violet) for about 45 seconds, rinsed with distilled water (without removing the fixed chicory and bacteria), a drop of immersion oil was added directly to the stained material, and the slide was examined under a light microscope at about 1000 to 2000 times magnification. Bacterial cells within 50 nanometers of the chicory were considered adhered, while bacterial cells greater than 50 nanometers from the chicory were considered non-adhered. The results were determined on one sample and counted using photomicrographs (taken from a Tucsen Imaging Technology, TSview7, 9 mp camera). The photomicrographs were captured and saved on a computer (Toshiba laptop), and color, light, contrast, and sharpness adjusted to the original image directly from the microscope (using Adobe® Photoshop Elements 9 or Google® Picasa 3). As shown in Table 3, results indicate the majority of bacterial cells observed (75.8%) were noted as attached to the chicory. Only 24.2% of the bacterial cells were unattached from the chicory.

TABLE 3
Results after Evaluating Pediococci under Light
Microscopy Using the Dry Stain Technique

| | | Total Number | Total Number |
|---|---|---|---|
| Obser- | Total Number | of Pediococci | of Pediococci Not |
| vation | of Pediococci | Attached to Chicory | Attached to Chicory |
| 1 | 132 | 100 (75.8%) | 32 (24.2%) |

Adhesion of a bacteria and/or yeast to a prebiotic due to a biofilm provides many advantages. While not wishing to be bound by theory, the present teachings believe that biofilm protects the bacteria from damage, degradation, and death when traveling in the intestinal tract of a host. This facilitates protection of the bacteria from various harmful components within the digestive tract, including ptyalin (digestive enzyme) in the mouth, hydrochloric acid secretion into the stomach and small intestine, bile acid secretion into the small intestine (duodenum), digestive enzyme secretions into the small intestine. Likewise, by attaching a prebiotic to a bacteria (and/or yeast), the bacteria may have a food source that is delivered to the same destination as the bacteria. Further, the presence of a biofilm may further enable attachment of the bacteria to the substrate of a host (e.g., the intestinal villi). Further still, a combiomic may facilitate site-specific delivery of a bacteria and/or yeast. Thus, by including a bacteria and/or yeast that adheres to a prebiotic, a combiomic complex may provide the advantage of aiding in the development of a favorable oral, intestinal and/or cecal/colonic microbiota when ingested by an animal or human. Indeed, given the magnitude of the prebiotic size relative to a probiotic (which may be in the form of a bacteria and/or yeast), the prebiotic particles provide protection to the probiotic through various parts of the intestinal tract. Thus, a combiomic complex provides for higher survival rates of live probiotic cells to targeted activation sites.

### Example 2

### Enhanced Resistance to Hydrochloric Acid of a Bacteria in a Combiomic Complex

In the embodiment of Example 2, enhanced survivability of a bacteria in a combiomic complex is experimentally shown. To this end, FIG. 5 is a bar graph 500, according to one preferred embodiment of the present teachings, showing the enhanced resistance of a bacteria, when exposed to hydrochloric acid (HCl) for 30 minutes, in a combiomic complex. FIG. 5 includes an x-axis 502 showing three types of test samples, (1) bacteria in a combiomic complex (i.e., bacteria adhered to a prebiotic), (2) bacteria in a pre-combiomic mixture (i.e., bacteria not adhered to a prebiotic, i.e., synbiotic), and (3) bacteria alone (i.e., bacteria not in the presence of a prebiotic). FIG. 5 also includes a y-axis 504, which shows growth of bacteria, as measured in CFU (log₁₀/g). As shown in FIG. 5, dashed bars shown growth of bacteria prior to exposure to HCl, and solid bars shows growth of bacteria after exposure to HCl for 30 minutes. As shown by the dashed bars in FIG. 5, all samples, i.e., combiomic, symbiotic, and probiotic alone, show similar microbial activity under treatment conditions without HCl. However, as shown by the solid bars, after thirty minutes of exposure to HCl, an acid typically present in the stomach, the bacteria in the combiomic sample retained the greatest amount of microbial activity, while bacteria in the synbiotic sample produced microbial activity greater than the sample of probiotic alone. Thus, FIG. 5 shows that bacteria adhered to a probiotic was best able to tolerate acid associated with the stomach environment as compared to bacteria and prebiotic that are not adhered, and bacteria alone.

Table 4 and Table 5 show compositions of a first fermented mixture and a second fermented mixture, respectively, used to generate the samples tested in FIG. 5.

TABLE 4
Composition Used to Create First “Fermented Mixture”

| Ingredient | Percent by Weight |
|---|---|
| Beef broth | To 100 |
| Dextrose | 1 |
| Starter Culture* | 0.0626 |

*Starter Culture contains sufficient Pediococcus acidilactici and Pediococcus pentosaceus to provide 1 × 10⁷ cfu/ml of beef broth. The concentration, 0.0625%, is an approximate concentration. As the concentration of bacteria in the starter culture may vary, the actual percentage included in the Combiomic Mixture may vary.

TABLE 5
Composition Used to Create Second “Fermented Mixture”

| Ingredient | Percent by Weight |
|---|---|
| Beef broth | To 100 |
| Dextrose | 1 |
| Chicory | 1 |
| Starter Culture* | 0.0626 |

*Starter Culture contains sufficient Pediococcus acidilactici and Pediococcus pentosaceus to provide 1 × 10⁷ cfu/ml of beef broth. The concentration, 0.0625%, is an approximate concentration. As the concentration of bacteria in the starter culture may vary, the actual percentage included in the Combiomic Mixture may vary.

Table 4 sets forth the composition of ingredients used to produce a “first fermented mixture,” which was generated by adding dextrose (i.e., energy source) to Swanson® Beef Broth (i.e., growth media) to achieve an approximately 1%, by weight, solution of dextrose in solution. Next, strains of Pediococcus acidilactici and Pediococcus pentosaceus (i.e., non-adhered bacteria) were inoculated to achieve a concentration of 1×10⁷ cfu/ml of beef broth. This mixture was then incubated at about 41° C. for about 24 hours to produce a “first fermented mixture.” The first fermented mixture was used as a source to obtain a bacteria alone sample and a pre-combiomic sample.

Table 5 sets forth the composition of ingredients used to produce a “second fermented mixture.” The second fermented mixture was generated in a substantially similar manner as described above with reference to the first fermented mixture. Generating the second fermented mixture, however, included the further step of adding ground chicory (Chicory Root Raw C/S Organic; Starwest Botanicals, Inc., Rancho Cordova, Calif.) (i.e., prebiotic) to create a 1%, by weight, of prebiotic in the second fermented mixture. The second fermented mixture was used as a source to obtain a combiomic complex sample.

The solid and liquid components of the first combiomic mixture and the second combiomic mixture were separated by decanting. The liquid supernatant of the first combiomic mixture was used as the source of the “bacteria-alone” sample, the liquid supernatant of the second fermented mixture was used as the source of the “pre-combiomic” sample, and the solid portion of the second fermented mixture was used as the source of the “combiomic” sample.

Approximately 1 ml of each of the bacteria-alone and pre-combiomic sources, and approximately 1 g of the combiomic source, were added to separate portions of about 99 ml of “acidified” phosphate buffered water. (The “acidified” phosphate buffered water was created by adjusting about 90 ml of stock solution Butterfield's buffered phosphate diluent to a pH of about 1.5 with about 0.1 ml HCl.) For the pre-combiomic sample, an additional about 0.2 g of chicory was also added. The samples equilibrated at about 27° C. for about 30 min About 1 ml of each of the resulting mixtures was removed and placed into about 9 ml Butterfield's buffered phosphate diluent to make separate approximately 1/10 dilutions, which were plated on MRS agar (Difco Laboratories, Inc., Detroit, Mich.). The plates were incubated at about 37° C. for between about 24 and 48 hours, and bacterial populations were then counted.

As shown in FIG. 5, within 30 minutes of exposure to HCl, approximately 1.3 logs of bacteria from the combiomic sample were deactivated, approximately 2.5 logs of bacteria from the pre-combiomic mixture were deactivated, and little to no survival (<10,000 cfu/g solution; 4 logs) was shown in the bacteria-only sample. Thus, according to the embodiment of FIG. 5, a bacteria that is adhered to a prebiotic is more resistant to treatment with HCl than a bacteria mixed with (but not adhered to) a prebiotic, or a bacteria alone.

### Example 3

### Treating of a Pre-Combiomic Mixture Carried Out in Multiple Steps

In the embodiment of Example 2, treating is carried out in multiple steps that may require separate treating conditions. To this end, Table 6 shows a composition used to generate a “fermented mixture,” according to one embodiment of the present teachings, that does not include a prebiotic in an initial treating step.

TABLE 6
Composition Used to Create “Fermented Mixture”

| Ingredient | Percent by Weight |
|---|---|
| Beef broth | To 100 |
| Dextrose | 1 |
| Starter Culture* | 0.0626 |

*Starter Culture contains sufficient Pediococcus acidilactici and Pediococcus pentosaceus to provide 1 × 10⁷ cfu/ml of beef broth. The concentration, 0.0625% by weight of the Fermented Mixture, is an approximate concentration. As the concentration of bacteria in the starter culture may vary, the actual percentage included in the Fermented Mixture may vary.

As shown in Table 6, Pediococcus acidilactici and Pediococcus pentosaceus (non-adhered bacteria) were inoculated in beef broth (Swanson® Beef Broth) (growth medium) to achieve about 1×10⁷ cfu/ml concentration of bacteria. Dextrose (energy source) was then added to the inoculated beef broth to achieve a 1% solution, by weight, of dextrose in solution. This mixture was then incubated for about 48 hours at about 41° C., after which it is referred to as a “fermented mixture.” Chicory (prebiotic) was then added to this fermented mixture to form a pre-combiomic mixture. The pre-combiomic mixture with the added chicory was then incubated at about 35° C. for about another 8 hours. During this incubation, the pre-combiomic mixture was gently agitated about every two hours, which facilitated contact between the non-adhered bacteria and non-adhered prebiotic. Agitation was performed by placing the container with the pre-combiomic mixture on a shaker table. At the end of the incubation period, the liquid portion was decanted off of the solid portion. The solid portion, which contains the combiomic complex, was then collected and frozen (at about −20° C.) until further use as a source of attached bacteria (i.e., combiomic complex).

### Example 4

### Viability of a Preserved Combiomic Solid Residue

In the embodiment of Example 4, the viability of a preserved combiomic solid residue is experimentally shown. To this end, Table 7 shows a pre-combiomic composition used to produce a combiomic, Table 8 shows a fresh combiomic composition and a preserved combiomic composition, and Table 9 shows a comparison of the pH generated by fermentation of the fresh combiomic composition and preserved combiomic composition of Table 8. The present teachings recognize that to the extent a preserved combiomic remains viable after long-term storage, inoculating said preserved combiomic complex in growth media will lower the pH of the growth media due to production of lactic acid during fermentation. Table 9 demonstrates the viability of a preserved combiomic complex that is stored for nine months.

TABLE 7
Composition Used to Create Combiomic Complex

| Ingredient | Percent by Weight |
|---|---|
| Beef broth | To 100 |
| Apple juice concentrate | 1 |
| Starter Culture* | 0.0626 |

*Starter Culture contains sufficient Pediococcus acidilactici and Pediococcus pentosaceus to provide 1 × 10⁷ cfu/ml of beef broth. The concentration, 0.0625%, is an approximate concentration. Since the concentration of bacteria in the starter culture can vary, the actual percentage included may vary.
**Apple juice concentrate was added to the beef broth to provide monosaccharides at 1% of the composition. As a result, apple juice concentrate was added at 36% of the mixture to achieve the addition of 1% monosaccharides from apple juice concentrate.

TABLE 8
Composition of Fresh and Preserved Combiomic Complex

| Fresh Combiomic Mixture¹ | | Preserved Combiomic Mixture² | |
|---|---|---|---|
| | Percent | | Percent |
| Ingredient | by Weight | Ingredient | by Weight |
| Combiomic (freshly | 2.42 | Combiomic | 1.37 |
| decanted supernatant) | | | |
| Whole ground | 96.59 | Glycerol | 30 |
| chicken meat | | | |
| Dextrose | 1 | Whole ground | 68.63 |
| | | chicken meat | |

¹Used combiomic complex immediately after making to make combiomic mixture.
²Combiomic complex added to glycerol and stored at −20° C for 8.5 months.

TABLE 9
pH of Meat Inoculated with Fresh
and Preserved Combiomic Complex

| Fresh Combiomic Mixture¹ | | Preserved Combiomic Mixture² | |
|---|---|---|---|
| Timepoint (conditions) | pH | Timepoint (conditions) | pH |
| Time 0 (immediately | 6.22 | Time 0 (stored | 6.85 |
| after making) | | combiomic removed | |
| | | and thawed to 22° C.) | |
| 12 h at 22° C. | 5.89 | 12 h at 22° C. | 5.1 |

¹Combiomic complex and meat mixture analyzed immediately after making.
²Combiomic complex added to glycerol and stored at −20° C for 8.5 months was removed from storage and allowed to thaw to ambient conditions and added to 75 g of chicken meat with dextrose added.

To generate the composition used in Table 9, about 300 ml of beef broth (Swanson® Beef Broth) with about 7.5 g of apple juice concentrate (Kroger® Apple Juice) was incubated for about 24 h at about 41° C. Apple juice concentrate was added to the beef broth (Swanson® Beef Broth) to achieve a 2.5% solution, by volume. Pediococcus acidilactici and Pediococcus pentosaceus were then inoculated in the beef broth to achieve about 1×10⁷ cfu/ml of beef broth. Chicory was also added to the inoculated beef broth and apple juice concentrate to achieve a 1% solution, by weight, of monosaccharides from apple juice concentrate. The mixture was then incubated for about 24 hours at about 41° C. The resulting combiomic composition was harvested by decanting the supernatant from the solid combiomic residue.

As shown in the compositions of Table 8, about 2.42 g of the solid combiomic residue was then added to about 100 g of whole ground chicken meat and dextrose to create a fresh combiomic complex. As shown in Table 9, the pH of the fresh combiomic complex was evaluated immediately (about 0 h) and about 12 hours later (Table 9). Separately, about 1.37 g of combiomic was added to about 68.63 g of whole ground chicken with glycerol (preservative) added to create about a 30% by weight of a combiomic mixture (Table 8). This combiomic mixture was stored at about −20° C. for about 8.5 months. After completing the storage time, this mixture was removed from storage, warmed to ambient (about 22° C.) conditions and the pH of the mixture evaluated immediately (about 0 h) and about 12 hours later (Table 9). As Table 9 demonstrates, the combiomic mixture that was stored in about 30% glycerol, by volume of the combiomic mixture, at about −20° C. for about 8.5 months, was able to reduce the pH of the mixture after about 12 hours of fermentation at ambient conditions as much as the freshly made combiomic mixture. As a result, storage of the combiomic under these conditions is sufficient to assure its viability at the end of shelf-life.

The present teachings disclose steps to produce a shelf-stable meat. First Pediococci, a source of bacteria were attached to chicory, a prebiotic source. About 2% Dextrose and about 0.1% Tween 80 were added to chicken broth that was then pasteurized to about 190° F. to create a pasteurized broth mixture. This mixture was heated to boiling (212° F.) and then cooled. When the mixture was about 180° F., 0.25% (w/v) finely ground dried chicory was added to the cooled broth mixture. After further cooling the cooled broth mixture to 125° F., Pediococcus acidilactici and P. pentosaceus were inoculated at the level of 1×10⁷ cfu/g to create an inoculated broth. The inoculated broth was placed in an environment at about 35° C. After about 72 hours, the liquid portion of the fermented broth was decanted off. The remaining moist precipitate containing the Pediococci attached to the chicory was then stored at refrigeration (4° C.) until use.

Next, shelf-stable chicken meat was developed by the following methods. The following were added and mixed into 100 g of whole ground chicken meat (pH=6.35): 1) 10 g of the moist precipitate stored at refrigeration described in Example 1; and 2) an E. coli (strains ATCC BAA 1427, 1428, 1429, 1430 and 1431) at the level to create a 1.4×10⁸ cfu/g concentration of E. coli on culture-inoculated whole ground chicken meat. The added moist precipitate served as a source of Pediococci that could decrease pathogen infestation and as well provide a source of shelf-stabilizing properties. The moist precipitate further provided a food source preferred by the Pediococci to enable its growth as well as protect the Pediococci to enable their ability to start growth in the meat. The added E. coli served as a pathogen source. The inoculated chicken meat was incubated at about 120° F. After 24 hours and 36 hours the fermented chicken meat pH and E. coli concentration was assessed. After 24 hour, the pH was 4.5 and the concentration of E. coli was 6.026×10⁶ cfu/g per gram of fermented whole chicken meat. After 48 h, the pH was 4.4 and the concentration of E. coli was 2.29×10⁵ cfu/g of fermented whole ground chicken meat.

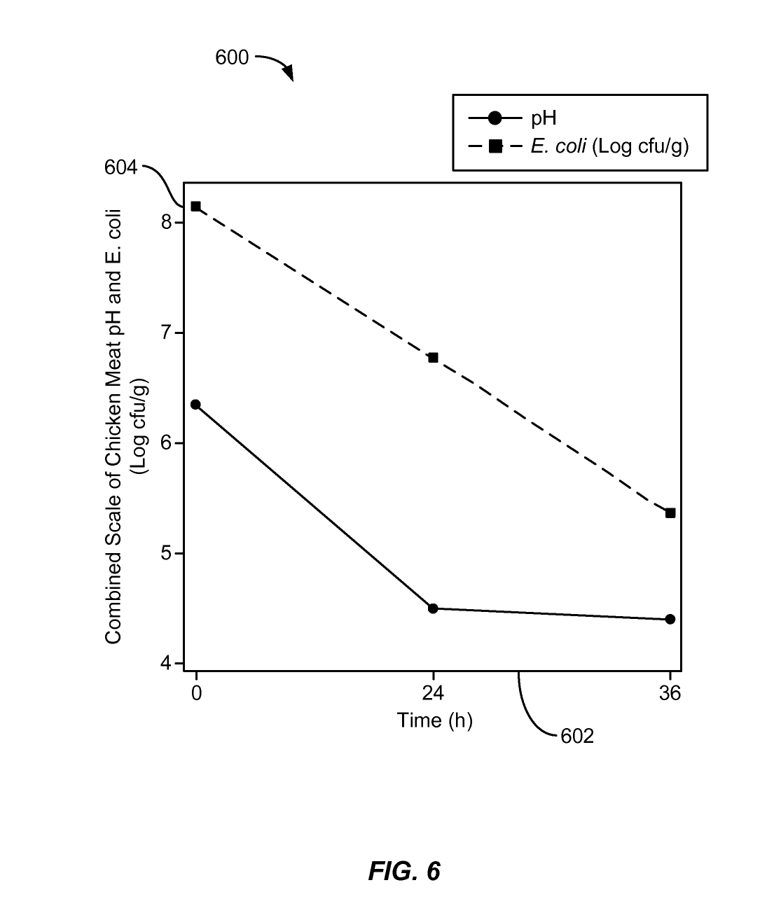

FIG. 6 is a graph showing a decrease in pathogenic activity on a chicken meat by incubating a combiomic complex having Pediococci adhered to chicory on the chicken meat to lower a pH of the chicken meat, according to the embodiment of example 4. FIG. 6 includes an x-axis 702, showing treatment time, a y-axis 704 showing pathogen growth (dashed line) and pH (solid line) at different treatment times. As shown in FIG. 6, the pH and concentration of E. coli dropped considerably indicating that the addition of Pediococci attached to chicory to the chicken meat was reduced in the contamination of E. coli and at a pH level indicative of a shelf-stable food product.

### Example 5

### Compatibility of a Health-Promoting Bacteria and a Pathogen-Controlling Bacteria in a Combiomic Complex

The compatibility of bacteria that promote health with bacteria that promote food stability in a combiomic complex is experimentally shown. Three fermented growth cultures were prepared as follows. About 2% Dextrose and about 0.1% Tween 80 was added to chicken broth that was then pasteurized to about 212° F. to create a pasteurized broth mixture. When the pasteurized broth mixture had cooled to about 180° F., threonine in the amount of about 0.1% by weight of amino acid enriched broth mixture, serine in the amount of about 0.05% by weight of amino acid enriched broth mixture, proline in the amount of about 0.05% by weight of amino acid enriched broth mixture, cysteine in the amount of about 0.1% by weight of amino acid enriched broth mixture were added to create an amino acid enriched broth mixture. The amino acid enriched broth mixture was further cooled to about 125° F. Then three different inoculated broth mixtures were made by inoculating them with different culture sources. Inoculated broth mixtures included: (1) addition of 1.0 g of a freeze dried mixture of Pediococcus acidilactici and P. pentosaceus to 200 ml of inoculated broth to create a Pediococci-only broth; (2) addition of 1.0 g of freeze dried Lactobacillus plantarum to 200 ml of inoculated broth to create a Lactobacillus-only broth; and (3) addition of 0.5 g of a freeze dried mixture of Pediococcus acidilactici and P. pentosaceus and 0.5 g of freeze dried Lactobacillus plantarum to 200 ml of inoculated broth to create a Pediococci/Lactobacillus broth. Each culture was added to create an inoculated broth with a bacteria concentration of about 1×10⁷ cfu/ml of inoculated broth.

The inoculated broth mixtures were placed in sealed containers and then placed in an environment at about 35° C. for about 48 hours to allow them to ferment to create fermented growth cultures. After 48 hours the fermented growth cultures were removed from the 35° C. environment and kept at ambient temperature until further use.

Three live-bacterial concentrates were prepared as follows. A sample of each of the three fermented growth cultures as previously described were stained with crystal violet for about 45 seconds, then gently rinsed with water, and finally visually observed using a light microscope at 1000 times magnification in order to estimate the population density. The light microscope was used to examine the field of three random samples obtained from each fermented growth culture. Each microscope field was counted for rods and diplococci and the ratio of dipliococci to rods using a Hausner Counting Chamber. All three fermented growth cultures were confirmed to have a bacterial concentration of about 1×10⁹ cfu/ml of fermented growth culture. Further, the fermented growth culture resulting from the Pediococci/Lactobacillus broth was confirmed to have about 1:1 ratio of Pediococci:Lactobacillus cultures.

The bacterial cells were recovered from each of the fermented growth cultures by centrifuging 200 ml of fermented growth culture at 2,500 RPM for 15 min. The supernatant was discarded and the recovered bacterial cells were re-suspended in about 50 ml of Butterfield's Phosphate Buffered Diluent (BPBD) at about 1×10¹⁰ cfu/g of BPVD to create live bacterial cell concentrates. The live bacterial cell concentrates were concentrated four times (4×), as 200 ml of fermented growth culture was used to obtain the recovered cells that were then reconstituted with 50 ml of BPBD. The live bacterial cell concentrates from each of the fermented growth cultures were stored in refrigeration at about 4° C. The three live bacterial cell concentrates are referred to as: (1) live Pediococci-only bacterial cell concentrate, (2) live Lactobacillus-only bacterial cell concentrate, and (3) live Pediococci/Lactobacillus bacterial cell concentrate.

Determination of the bacterial killing ability of live pre-combiomics was also conducted. In this example, a microtiter well experiment was conducted to evaluate the efficacy of the live bacterial cell concentrates as previously described on killing and inhibition of E. coli (ATCC BAA strains 1427, 1428, 1429, 1430, 1431). The process to evaluate this using a microtiter plate is described as follows. About 100 μl of chicken broth was added to wells 2 through 12 of a microtiter plate. About 100 μl (about 1×10⁷ cfu) of the live Pediococci bacterial cell concentrate and about 100 μg of finely ground chicory was added into wells 1 and 2. The addition of chicory to Pediococci creates a pre-combiomic composition, as the chicory is used as an energy source by the Pediococci bacteria. The combination of the live Pediococci bacterial cell concentrate and chicory is referred to as live Pediococci pre-combiomic composition.

Well 1 served as a control as no chicken broth was added. 100 μl was drawn from well 2 and placed into well 3. For well 3 and thereafter repeated, 100 μl samples were drawn and placed into the next well to result in the following dilutions for wells 1 through 12: about 1:1, about 1:2, about 1:4, about 1:8, about 1:16, about 1:32, about 1:64, about 1:128, about 1:256, about 1:512, about 1:1024, and about 1:2048. The previously described procedure for wells 1 through 12 was repeated in a second and third set of microtiter wells using the other two live bacterial cell concentrates. The combination of the live Lactobacillus-only bacterial cell concentrate and chicory is referred to as live Lactobacillus-only pre-combiomic composition. The combination of the live Pediococci/Lactobacillus bacterial cell concentrate and chicory is referred to as live Pediococci/Lactobacillus pre-combiomic composition.

About 100 μl of E. coli (ATCC BAA strains 1427, 1428, 1429, 1430, 1431) solution that provided about 1×10⁷ cfu was added to wells 1 through 12 of each live bacterial cell concentrate. About 100 μl of chicken broth and about 100 μl of E. coli (ATCC BAA strains 1427, 1428, 1429, 1430, 1431) solution that provided about 1×10⁷ cfu were added into an additional microtiter plate well that served as a positive control (to demonstrate that the E. coli would grow without the addition of the live bacterial cell concentrates).

The microtiter plate was covered and placed in an incubator at about 35° C. for about 48 hours. After the appropriate incubation time the microtiter plate was removed from the incubator. About 100 μl of the metabolic indicator iodonitrotetrazolium chloride was added to each well. The microtiter plate was placed into the 35° C. incubator for about 2 hour to develop the color change. Upon removal from the incubator, the results were recorded.

Results are displayed in Table 10. Results indicate that pathogen killing power occurred equally with all three live pre-combiomic compositions: (1) live Pediococci-only pre-combiomic composition, (2) live Lactobacillus-only pre-combiomic composition, and (3) live Pediococci/Lactobacillus pre-combiomic composition. Further, all three live pre-combiomic compositions, regardless of the bacterial source had about the same killing power against E. coli.

Results also indicate that the addition of Lactobacillus to a blend of Pediococci results in a compatible mixture of organisms to provide food product shelf-stability, Pediococci, and those organisms with expected health benefits, Lactobacillus.

TABLE 10
Results Comparing The Pathogen Killing
Power Of Live Synbiotics

| | | Live | Live | Live |
|---|---|---|---|---|
| | | Pediococci- | Lactobacillus- | Pediococci/ |
| | | Only Pre- | Only Pre- | Lactobacillus |
| Well # | Dilution | Combiomic | Combiomic | Pre-Combiomic |
| 1 | 1:1 | − | − | − |
| 2 | 1:2 | − | − | − |
| 3 | 1:4 | + | + | + |
| 4 | 1:8 | + | + | + |
| 5 | 1:16 | + | + | + |
| 6 | 1:32 | + | + | + |
| 7 | 1:68 | + | + | + |
| 8 | 1:128 | + | + | + |
| 9 | 1:256 | + | + | + |
| 10 | 1:512 | + | + | + |
| 11 | 1:1024 | + | + | + |
| 12 | 1:2048 | + | + | + |
| | No | ++ | ++ | ++ |
| | Concentrate | | | |

+ = Growth
−1 = Inhibition
− = No Growth

### Example 6

### Superior Efficacy of Combiomic Complexes Over Pre-Combiomic Compositions

Combiomic complexes are shown to be more effective than pre-combiomic compositions in the following examples.

The development of three different bacterial cultures attached to a prebiotic was performed as follows. About 2% Dextrose and about 0.1% Tween 80 were added to chicken broth that was then pasteurized to about 212° F. to create a pasteurized broth mixture. When the pasteurized broth mixture had cooled to about 180° F., threonine in the amount of about 0.1% by weight of amino acid enriched broth mixture, serine in the amount of about 0.05% by weight of amino acid enriched broth mixture, proline in the amount of about 0.05% by weight of amino acid enriched broth mixture, cysteine in the amount of about 0.1% by weight of amino acid enriched broth mixture were added to create an amino acid enriched broth mixture. To about 200 ml of the amino acid enriched broth mixture, about 4 grams of finely ground chicory was added to create a prebiotic enriched broth mixture. The prebiotic enriched broth mixture was further cooled to about 125° F. Then, three different inoculated prebiotic broth mixtures were made by inoculating them with different culture sources. Inoculated prebiotic broth mixtures included: (1) addition of about 1.0 g of a freeze dried mixture of Pediococcus acidilactici and P. pentosaceus to about 200 ml of prebiotic enriched broth mixture to create a Pediococci-only prebiotic broth; (2) addition of about 1.0 g of freeze dried Lactobacillus plantarum to about 200 ml of prebiotic enriched broth mixture to create a Lactobacillus-only prebiotic broth; and (3) addition of about 0.5 g of a freeze dried mixture of Pediococcus acidilactici and P. pentosaceus and about 0.5 g of freeze dried Lactobacillus plantarum to about 200 ml of prebiotic enriched broth mixture to create a Pediococci/Lactobacillus prebiotic broth. Each culture was added to create an inoculated broth with a bacteria concentration of about 1×10⁷ cfu/ml of inoculated broth.

The inoculated broth mixtures were placed in sealed containers and then placed in an environment at about 35° C. for about 48 hours to allow them to ferment to create prebiotic-attached bacterial cultures. After about 48 hours, the prebiotic-attached bacterial cultures were removed from the 35° C. environment and kept at ambient temperature until further use.

Development of three combiomic cultures is shown by the following example. A sample of each of the three prebiotic-attached bacterial cultures described in the previous example were stained with crystal violet for about 45 seconds, then gently rinsed with water, and finally visually observed using a light microscope at 1000× magnification in order to estimate the population density. The light microscope was used to examine the field of three random samples obtained from each fermented growth culture. Each microscope field was counted for rods and diplococci and the ratio of dipliococci to rods using a Hausner Counting Chamber. All three prebiotic-attached bacterial cultures were confirmed to have a bacterial concentration of about 1×10⁹ cfu/ml of fermented growth culture. Further, the prebiotic-attached bacterial culture resulting from the Pediococci/Lactobacillus broth was confirmed to have about 1:1 ratio of Pediococci:Lactobacillus cultures.

The prebiotic-attached bacterial cells were recovered from each of the prebiotic-attached bacterial cultures by centrifuging about 200 ml of prebiotic-attached bacterial cultures at about 2,500 RPM for about 15 min. The supernatant was discarded and the recovered prebiotic-attached bacterial cells were resuspended in about 50 ml of Butterfield's Phosphate Buffered Diluent (BPBD) at about 1×10¹⁰ cfu/g of BPBD to create combiomic 4× concentrates. The combiomic 4× concentrates were concentrated four times (4×), as about 200 ml of fermented growth culture was used to obtain the recovered cells that were then reconstituted with about 50 ml of BPBD. The combiomic 4× concentrates from each of the prebiotic-attached bacterial cultures were stored in refrigeration (about 4° C.). The three combiomic 4× concentrates are referred to as: (1) Pediococci-only combiomic 4× concentrate; (2) Lactobacillus-only combiomic 4× concentrate; and 3) Pediococci/Lactobacillus combiomic 4× concentrate.

In the following example, the bacterial killing ability of combiomic 4× concentrates is shown. In this example, a microtiter well experiment was conducted to evaluate the efficacy of the combiomic 4× concentrates as described in Example 7 on killing E. coli (ATCC BAA strains 1427, 1428, 1429, 1430, 1431). The process to evaluate this using a microtiter plate is described as follows. About 100 μl of chicken broth was added to wells 2 through 12 of a microtiter plate. About 100 μl (about 1×10⁷ cfu) of the Pediococci-only combiomic 4× concentrate was added into wells 1 and 2.

Well 1 served as a control as no chicken broth was added. 100 μl was drawn from well 2 and placed into well 3. For well 3 and thereafter repeated, 100 μl samples were drawn and placed into the next well to result in the following dilutions for wells 1 through 12: 1:1, 1:2, 1:4, 1:8, 1:16, 1:32, 1:64, 1:128, 1:256, 1:512, 1:1024, and 1:2048. The previously described procedure for wells 1 through 12 was repeated in a second and third set of microtiter wells using the other two combiomic 4× concentrates.

About 100 μl of E. coli (ATCC BAA strains 1427, 1428, 1429, 1430, 1431) solution that provided about 1×10⁷ cfu was added to wells 1 through 12 of each combiomic 4× concentrate. About 100 μl of chicken broth and about 100 μl of E. coli (ATCC BAA strains 1427, 1428, 1429, 1430, 1431) solution that provided about 1×10⁷ cfu were added into an additional microtiter plate well that served as a positive control (to demonstrate that the E. coli would grow without the addition of the combiomic 4× concentrates).

The microtiter plate was covered and placed in an incubator at about 35° C. for about 48 hours. After the appropriate incubation time the microtiter plate was removed from the incubator. About 100 μl of the metabolic indicator iodonitrotetrazolium chloride was added to each well. The microtiter plate was placed into the 35° C. incubator for about 2 hours to develop the color change. Upon removal from the incubator, the results were recorded.

Results are shown in Table 11. Results indicate that pathogen killing power ranked from greatest to least was as follows: (1) Pediococci/Lactobacillus combiomic 4× concentrate; (2) Pediococci-only combiomic 4× concentrate; and (3) Lactobacillus-only combiomic 4× concentrate. Results suggest a synergistic benefit of the combined Pediococci/Lactobacillus combiomic 4× concentrate compared to either the Pediococci-only or Lactobacillus-only combiomic 4× concentrate.

Further, when results noted in Table 11 are compared to those in Table 10, it is apparent that combiomic complexes are more effective than pre-combiomic compositions. This is evidenced by the fact that both the Pediococci-only combiomic 4× concentrate and the Pediococci/Lactobacillus combiomic 4× concentrate resulted in greater pathogen kill (see Table 11) compared to live Pediococci-only pre-combiomic and live Pediococci/Lactobacillus pre-combiomic (see Table 10).

TABLE 11
Results Comparing the Pathogen Killing
Power of Combiomic 4x Concentrates

| | | Pediococci- | Lactobacillus- | Pediococci/ |
|---|---|---|---|---|
| | | Only | Only | Lactobacillus |
| | | Combiomic 4X | Combiomic 4X | Combiomic 4X |
| Well # | Dilution | Concentrate | Concentrate | Concentrate |
| 1 | 1:1 | − | − | − |
| 2 | 1:2 | − | − | − |
| 3 | 1:4 | −1 | + | −1 |
| 4 | 1:8 | + | + | −1 |
| 5 | 1:16 | + | + | + |
| 6 | 1:32 | + | + | + |
| 7 | 1:68 | + | + | + |
| 8 | 1:128 | + | + | + |
| 9 | 1:256 | + | + | + |
| 10 | 1:512 | + | + | + |
| 11 | 1:1024 | + | + | + |
| 12 | 1:2048 | + | + | + |
| | No | ++ | ++ | ++ |
| | Concentrate | | | |

+ = Growth
−1 = Inhibition
− = No Growth

As used herein, the articles including “the”, “a” and “an” when used in a claim or in the specification, are understood to mean one or more of what is claimed or described. As used herein, the terms “include”, “includes,” and “including” are meant to be non-limiting. All percentages and ratios are calculated and provided by weight unless otherwise indicated. All percentages and ratios are calculated and provided based on the total composition unless otherwise indicated.

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

Although illustrative embodiments of the present teachings have been shown and described, other modifications, changes, and substitutions are intended. Accordingly, it is appropriate that the appended claims be construed broadly and in a manner consistent with the scope of the disclosure, as set forth in the following claims.

What is claimed:

1. A process for producing a combiomic complex, said process comprising:
obtaining a pre-combiomic mixture, which includes at least one non-adhered bacteria and/or yeast and at least one non-adhered prebiotic;
treating at least some of said pre-combiomic mixture to form a combiomic mixture, which comprises a combiomic complex that includes at least one of said bacteria and/or said yeast adhered to at least one prebiotic.

2. The process for producing a combiomic complex of claim 1, wherein said obtaining said pre-combiomic mixture includes obtaining between about 1×10⁵ and about 1×10¹⁰ colony forming units (“CFU”) of bacteria and/or yeast per gram of said non-adhered prebiotic.

3. The process for producing a combiomic complex of claim 1, wherein said treating facilitates secretion of a biofilm by said non-adhered bacteria and/or yeast, such that said biofilm promotes adhering of said non-adhered bacteria and/or said yeast to said non-adhered prebiotic.

4. The process for producing a combiomic complex of claim 1, further comprising removing said combiomic complex from said combiomic mixture.

5. The process for producing a combiomic complex of claim 4, further comprising drying said combiomic complex.

6. The process for producing a combiomic complex of claim 1, wherein an amount of combiomic complex is a value that is between about 0.0001% by weight of combiomic mixture and about 10% by weight of combiomic mixture.

7. The process for producing a combiomic complex of claim 1, further comprising placing said bacteria and/or said yeast in said combiomic complex, which is in a non-fermenting state.

8. A combiomic complex composition comprising a combiomic complex that includes at least one bacteria and/or said yeast adhering to at least one prebiotic, wherein at least one of said bacteria and/or said yeast in said combiomic complex composition is in a substantially non-fermenting state.

9. The combiomic complex composition of claim 8, wherein said combiomic complex is free of digestive enzymes found in a human or an animal.

10. The combiomic complex composition of claim 8, wherein said combiomic complex does not contact food.

11. The combiomic complex composition of claim 8, further comprising a biofilm that is produced during growth of at least one of said bacteria and/or said yeast and that facilitates adhesion of at least one of said bacteria and/or said yeast to at least one of said prebiotic.

12. The combiomic complex composition of claim 11, wherein said biofilm is an exopolysaccharide.

13. The combiomic complex composition of claim 11, wherein said biofilm includes at least one member chosen from a group comprising galacturonic acid, glucuronic acid, glycoproteins, and mannuronic acid.

14. The combiomic complex composition of claim 11, wherein said biofilm comprises a glycocalyx matrix that surrounds at least one of said bacteria and/or said yeast to facilitates adhesion of at least one of said bacteria and/or said yeast to at least one of said prebiotic.

15. The combiomic complex composition of claim 8, wherein said bacteria and/or said yeast includes at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Saccharomyces cerevisiae, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, and Oxalobacter formagenes.

16. The combiomic complex composition of claim 8, wherein said prebiotic includes at least one member chosen from a group comprising fructooligosaccharides (FOS), short-chain FOS, galactooligosaccharides, xylooligosaccharides, oligo derivatives from starch, inulin, chicory, soy oligosaccharides, trehalose, raffinose, stachyose, lactosucrose, lactulose, apple pomace, paw paw, galacto-oligosaccharides, soybean oligosaccharides, gluco-oligosaccharides, cyclodextrins, gentio-oligosaccharides, germinated barley foodstuffs, oligodextrans, pecti-oligosaccharides, mannan-oligosaccharides, lactose, resistant starches, oligo-saccharides, oligo-saccharides from melobiose, n-acetylchito-oligosaccharides, shrimp shells, polydextrose, sugar alcohols, konjac glucomannan, whole grain, corn steep, Jerusalem Artichoke, wheat bran, rice bran, plantain, bananas, apple pulp, B-glucan, Carboxymethylcellulose (CMC), Methylcellulose, Hydroxypropylmethylcellulose (HPMC), Psyllium, Guar gum, Citrus pectin, Pectin, Xanthan gum, Gum Arabic, Gum talha, and Alginate.

17. The combiomic complex composition of claim 8, wherein in said combiomic complex composition, a combiomic size ratio, which refers to a ratio of a particle size of at least one of said prebiotic to an individual cell size of at least one of said adhered bacteria and/or said yeast, is between about 200:1 and about 8000:1.

18. The combiomic complex composition of claim 8, further comprising a fluid, wherein a concentration of said combiomic complex in said combiomic complex composition is a value that is between about 0.0001% by weight of said combiomic complex composition and about 5% by weight of said combiomic complex composition.

19. The combiomic complex composition of claim 8, wherein said combiomic complex composition is in a state that is at least one member chosen from a group comprising solid dried, freeze-dried, powdered, granule, fluid, gel, liquid, solid, and syrup.

20. The combiomic complex composition of claim 8, further comprising at least one member chosen from a group comprising a palatant, a flavor enhancer, a food, a fat-enriched food or medicine, a binder, a preservative, an energy source, a beverage, a supplement, a pill, a coating, and a food coating.

21. The combiomic complex composition of claim 20, wherein said energy source is at least one member chosen from a group comprising apple juice, apple juice concentrate, dextrose, dextrose monohydrate, dextrose hydride, grape sugar, D-glucose, corn sugar, corn syrup solids, high fructose corn syrup, levulose, invert sugar, glucose, galactose, xylose, ribose, mannose, sorbose, high fructose corn syrup, apple pulp, honey, sugar, maple syrup, pear juice, grape juice, orange juice, and fruit juice.

22. The combiomic complex composition of claim 20, wherein said preservative includes at least one member chosen from a group comprising glycerol, dextrose, vitamin E, milk solids, sugar concentrates, propylene glycol, dimethyl sulfoxide (DMSO), mannitol, sorbitol, casein, meat concentrates, humectants, non-ionizing compounds that include many humectants, glycine betaine, sugars, sucrose, fructose, galactose, lactose, ethylene glycol, erythritol, threitol, dimethylformamide, 2-methyl-2,4-pentanediol, trehalose, tween 80, and capsular material.

23. The combiomic complex composition of claim 22, wherein an amount of said preservative present in said combiomic complex ranges from between about 30% by weight of said combiomic complex composition to about 70% by weight of said combiomic complex composition.

24. The combiomic complex composition of claim 8, further comprising a carrier for conveying said combiomic complex composition to a human or an animal and that is at least one member chosen from a group comprising capsule, sachet, pill, human food, pet food, pet treat, supplement, meat, dairy product, vegetable, fruit, fermented beverage, topical application, wound dressing, sunscreen, makeup product, mascara, cream, mouthwash, tampon, feminine pad, and dentifrice.

25. The combiomic complex composition of claim 24, wherein said carrier includes at least about 1×10⁵ cfu per gram of at least one of said bacteria and/or said yeast in said combiomic complex composition.

26. The combiomic complex composition of claim 8, wherein said bacteria and/or said yeast includes one species that promotes human or animal health and/or another species that promotes pathogen control in human or animal food.

27. The combiomic complex composition of claim 26, wherein said bacteria or said yeast that promotes human or animal health includes at least one member chosen from a group Lactobacillus, Bifidobacteria, Lactobacillus plantarum, Lactobacillus acidiophilis, Lactobacillus reuterii, Bifidobacterium bifidus, Pediococcus acidilactici, Pediococcus pentosaceus, and Bifidobacterium animalis.

28. The combiomic complex composition of claim 26, wherein said bacteria or said yeast that promotes pathogen control in a human or animal food includes at least one member chosen from a group comprising a Pediococci, Pediococcus pentosaceus, Pediococcus acidilactici, Lactococcus lactis, Lactococcus cremoris, Lactobacillus plantarum, Lactobacillus acidiophilis, Lactobacillus reuterii, L. bulgaricus, L. cuvatus, L. sakaii, L. fermentum, and Enterococcus faecium.

29. The combiomic complex composition of claim 8, wherein said combiomic complex composition is shelf-stable for a time that is at least about 6 months.

30. A pre-combiomic composition comprising:
at least one bacteria and/or yeast;
at least one prebiotic that is not adhering to said bacteria and/or said yeast; and
wherein concentration of at least one of said prebiotic is between about 0.0001% by weight of said pre-combiomic composition and about 2% by weight of said pre-combiomic composition, and wherein concentration of at least one of said bacteria and/or said yeast has between about 1×10⁵ cfu/g of said pre-combiomic composition and about 1×10⁸ cfu/g of said pre-combiomic composition.

31. The pre-combiomic composition of claim 30, further comprising a growth medium and a culture energy source.

32. A method for promoting human or animal health, said method comprising:
providing a combiomic complex that includes at least one bacteria and/or a yeast adhering to at least one prebiotic, wherein at least one of said bacteria and/or said yeast in said combiomic complex composition is in a substantially non-fermenting state; and
directing use of said combiomic complex by a human or an animal.

33. The method for promoting human or animal health of claim 32, wherein said promoting human or animal health includes treating at least one health condition chosen from a group comprising heart disease, dental caries, intestinal disorder, irritable bowel syndrome, diarrhea, acne, skin wrinkles, skin disease, gum disease, plaque formation, gum infection, inflammation, and improving healing during tooth removal.

34. The method for promoting human or animal health of claim 32, wherein said bacteria and/or said yeast that adheres to at least one of said prebiotic is more resistant than non-adhered bacteria and/or yeast to acid exposure.

35. The method for promoting human or animal health of claim 32, wherein said directing facilitates development of microbiota in at least one member chosen from a group comprising mouth, intestine, and colon that promotes human and/or animal health.

36. The method for promoting human or animal health of claim 32, wherein said providing includes preparing a topical application that includes said combiomic complex, and said directing includes providing instructions for applying said topical application on a surface of said human or said animal.

37. The method for promoting human or animal health of claim 36, wherein said topical application is at least one member chosen from a group comprising spray, wound dressing, sunscreen, makeup product, mascara, cream, mouthwash, tampon, feminine pad, and dentifrice.

38. The method for promoting human or animal health of claim 36, wherein said surface of said human or said animal includes at least one surface of a member chosen from a group comprising tooth, gum, skin, mucous membrane, and ear canal.

39. A method for promoting pathogen control in food, said method comprising:
obtaining a food item;
applying to said food item a combiomic complex to form a combiomic food product;
incubating said combiomic food product to form a substantially pathogen free food; and
wherein said combiomic complex includes at least one bacteria and/or a yeast that adheres to at least one prebiotic by virtue of a biofilm produced by at least one of said bacteria and/or said yeast.

40. The method for promoting pathogen control in food of claim 39, wherein said food is at least one member chosen from a group comprising ice cream, yogurt, milk, meat, fermented meat, kibbled food, kibble, an expanded food, pelleted food, extruded food, refrigerated food, refrigerated treat, frozen food, frozen treat, biscuit, raw food, fried food or treat, soft-moist food, pellet, fine, broken piece of food, jerky-style treat, injection-molded treat, treat, supplement, salad ingredient, ground fruit or vegetable, meal, slaughtered carcass, piece or chunk of meat, fabricated meat, fabricated protein chunk, livestock feed, steam-flaked feed, and aquaculture feed.

41. A method for producing a shelf-stable food, said method comprising:
obtaining a food item;
applying to said food item a combiomic complex to form a combiomic food product;
incubating said combiomic food product to form a substantially shelf-stable food; and
wherein said combiomic complex includes at least one bacteria and/or a yeast that adheres to at least one prebiotic by virtue of a biofilm produced by at least one of said bacteria and/or said yeast.

42. The method for producing a shelf-stable food of claim 41, wherein said shelf-stable food is substantially deprived of one or more food spoilage organisms selected from a group comprising Rhizopus nigricans, Penicillum, Aspergillus niger, Bacillus subtilis, Enterobacter aerogenes, Saccharomyces, Zygosaccharomyces, Micrococcus roseus, Aspergillus, Rhizopus, Erwinia, Botrytis, Rhodotorula, Alcaligenes, Clostridium, Proteus vulgaris, Pseudomonas fluorescens, Micrococcus, Lactobacillus, Leuconostoc, Alcaligenes, Flavobacterium, Proteus, and Acetobacter.
